(19) United States
(12) Patent Application Publication
(10) Pub. No.: US 2023/0141718 A1
(43) Pub. Date: May 11, 2023

(54) METHODS OF TREATING GLIOBLASTOMA

(71) Applicant: CELGENE QUANTICEL RESEARCH, INC., San Diego, CA (US)

(72) Inventors: Ellen FILVAROFF, San Francisco, CA (US); Bishoy HANNA, Summit, NJ (US); Manisha LAMBA, Summit, NJ (US); Ida ARONCHIK, Burlingame, CA (US)

(73) Assignee: CELGENE QUANTICEL RESEARCH, INC., San Diego, CA (US)

(21) Appl. No.: 17/983,732

(22) Filed: Nov. 9, 2022

Related U.S. Application Data
(60) Provisional application No. 63/277,976, filed on Nov. 10, 2021.

(51) Int. Cl. A61K 31/472; A61K 31/495; A61P 35/00

(52) U.S. Cl. CPC A61K 31/472; A61K 31/495; A61P 35/00

(57) ABSTRACT

The present application relates generally to methods for treating glioblastoma, or glioblastoma multiforme (GBM), with substituted heterocyclic derivative 4-[2-(cyclopropylmethoxy)-5-methylsulfonylphenyl]-2-methylisoquinolin-1-one, or the pharmaceutically acceptable salt thereof.

### RELATED APPLICATIONS

This application claims the benefits of priority of U.S. provisional application 63/277,976, filed Nov. 10, 2021, the entire contents of which are incorporated herein by reference.

### FIELD

The present application relates generally to compositions and methods for treating glioblastoma, or glioblastoma multiforme (GBM), with a substituted heterocyclic derivative compound, or a pharmaceutically acceptable salt thereof, as a bromodomain inhibitor.

### BACKGROUND

Glioblastoma, or glioblastoma multiforme (GBM), is the most common and aggressive malignant tumor found in the central nervous system, specifically the brain. Glioblastoma is derived specifically from the astrocyte cell type, in which the cellular growth is unregulated and tumor formation occurs. Initial symptoms of glioblastoma include headaches, dizziness, nausea, lethargy, seizures, hemiparesis, visual loss, stroke-like symptoms, memory problems, and personality changes.

Glioblastoma represents approximately 57% of all gliomas and 48% of all primary malignant central nervous system (CNS) tumors. It is estimated that more than 10,000 individuals in the United States will succumb to glioblastoma every year. Although multiple treatments have emerged in recent years, such as surgery, radiotherapy, and chemotherapy, the overall survival of patients with glioblastoma has not changed significantly for decades. At present, there are only two drugs approved by the US Food and Drug Administration (FDA) to treat glioblastoma via systematical administration: temozolomide (TMZ) for the treatment of newly diagnosed GBM (ndGBM) and bevacizumab for the treatment of recurrent GBM (rGBM). Unfortunately, current therapeutic approaches have very limited impact on improving the prognosis of glioblastoma patients, showing 15 months of median survival and less than 5% with a 5-year survival rate.

Temozolomide, sold as TEMODAR®, is classified as an alkylating anti-cancer agent. Temozolomide is indicated for newly diagnosed glioblastoma multiforms (GBM) concomitantly with radiotherapy and then as a maintenance treatment with no radiotherapy. Newly diagnosed GBM patients receive 75 mg/m² for 42 days concomitant with focal radiotherapy followed by a maintenance dose of 150 mg/m² temozolomide once daily for days 1-5 of a 28-day cycle. This maintenance dose cycle is then repeated for 6 cycles.

Differentiated from the alkylating agents, such as temozolomide, are several classes of other anticancer drugs which function at the epigenetic level, including inhibitors of DNA methyltransferase, histone deacetylase (HDAC), lysine-specific demethylase 1, zeste homolog 2, and bromodomain and extra-terminal motif (BET) proteins.

BET proteins have multiple functions, including the initiation and elongation of transcription and cell cycle regulation. In recent years, inhibitors of BET proteins have been developed as anticancer agents. These inhibitors exhibit selectivity for tumor cells by preferentially binding to superenhancers, noncoding regions of DNA critical for the transcription of genes that determine a cell's identity.

Thus, there remains a need for more effective treatments for glioblastoma, and this disclosure satisfies this need.

### SUMMARY

The present application relates generally to compositions and methods for treating glioblastoma.

Provided in one aspect is a method of treating glioblastoma multiforme (GBM) in a newly diagnosed GBM subject in need thereof comprising: (i) administering to the newly diagnosed GBM subject, a concomitant treatment of radiotherapy with temozolomide and a compound having the structure of Formula (I),

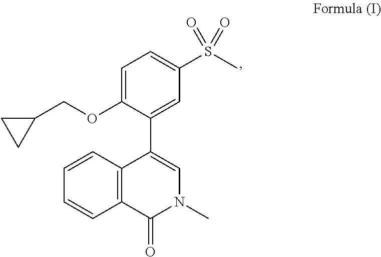 [SMILES: Cn1cc(-c2cc(S(C)(=O)=O)ccc2OCC2CC2)c2ccccc2c1=O]

or a pharmaceutically acceptable salt thereof; thereafter (ii) adjunctively administering temozolomide and the compound of Formula (I) or a pharmaceutically acceptable salt thereof without radiotherapy; and thereafter (iii) administering the compound of Formula (I) or a pharmaceutically acceptable salt thereof as a monotherapy.

In some embodiments, step (i) comprises a 42-day treatment regimen comprising administering the compound of Formula (I) or a pharmaceutically acceptable salt thereof on Days 1-4 and Days 35-39. In some embodiments, step (i) comprises a 42-day treatment regimen comprising administering 15 mg or 30 mg of the compound of Formula (I) or a pharmaceutically acceptable salt on Days 1-4 and Days 35-39. In some embodiments, step (i) comprises a 42-day treatment regimen comprising administering temozolomide once daily for the 42 days. In some embodiments, step (i) comprises a 42-day treatment regimen comprising administering 75 mg/m² of temozolomide once daily for the 42 days.

In some embodiments, step (ii) comprises a 28-day cycle comprising administering the compound of Formula (I) or a pharmaceutically acceptable salt thereof on Days 1-4. In some embodiments, step (ii) comprises a 28-day cycle comprising administering 15 mg, 30 mg, or 45 mg of compound of Formula (I) or a pharmaceutically acceptable salt thereof on Days 1-4. In some embodiments, step (ii) comprises a 28-day cycle comprising administering temozolomide on Days 1-5. In some embodiments, step (ii) comprises a 28-day cycle comprising administering 150 mg/m² of temozolomide on Days 1-4. In some embodiments, step (ii) comprises a 28 day cycle repeating from 2 to 6 times, and wherein 15 mg, 30 mg, or 45 mg of the compound of Formula (I) or a pharmaceutically acceptable salt is administered on Days 1-4 of cycles 2-6 and wherein temozolomide is administered at a dose of 200 mg/m² on Days 1-5 of cycles 2-6.

In some embodiments, step (iii) comprises a 28-day cycle comprising administering the compound of Formula (I) or a pharmaceutically acceptable salt thereof on Days 1-4. In some embodiments, step (iii) comprises a 28-day cycle comprising administering 45 mg of compound of Formula (I) or a pharmaceutically acceptable salt thereof on Days 1-4. In some embodiments, step (iii) comprises a 28-day cycle comprising administering the compound of Formula (I) or a pharmaceutically acceptable salt thereof is over one, two, three, four, five, six, seven, eight, nine, or ten cycles.

Provided in another aspect a method of treating glioblastoma multiforme (GBM) in a subject in need thereof comprising administering adjuvant therapy administering temozolomide and the compound of having the structure of Formula (I),

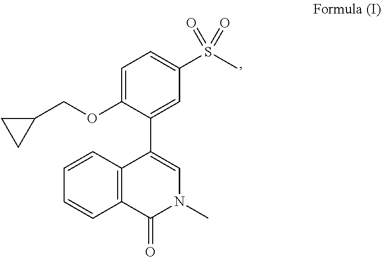 [SMILES: Cn1cc(-c2cc(S(C)(=O)=O)ccc2OCC2CC2)c2ccccc2c1=O]

or a pharmaceutically acceptable salt thereof without radiotherapy, and wherein administering the temozolomide and the compound of Formula (I) are in a 28 day cycle.

In some embodiments, administering the compound of Formula (I) or a pharmaceutically acceptable salt thereof is on Days 1-4. In some embodiments, 15 mg, 30 mg, or 45 mg of the compound of Formula (I) or a pharmaceutically acceptable salt is administered on Days 1-4. In some embodiments, administering temozolomide is on Days 1-5. In some embodiments, 150 mg/m² of temozolomide is administered on Days 1-4 of a 28-day cycle. In some embodiments, the 28-day cycle repeats 2 to 6 times, and wherein 15 mg, 30 mg, or 45 mg of the compound of Formula (I) or a pharmaceutically acceptable salt is administered on Days 1-4 of cycles 2-6, and wherein temozolomide is administered at a dose of 200 mg/m² on Days 1-5 of cycles 2-6.

Provided in another aspect is a method of treating glioblastoma multiforme (GBM) in a subject in need thereof comprising administering a monotherapy treatment of 45 mg of a compound having the structure of

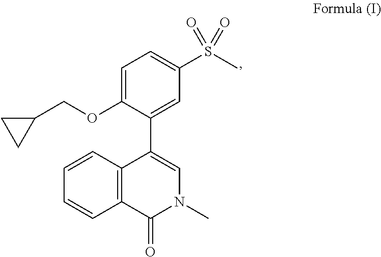 [SMILES: Cn1cc(-c2cc(S(C)(=O)=O)ccc2OCC2CC2)c2ccccc2c1=O]

or a pharmaceutically acceptable salt thereof on Days 1-4 of a 28 day cycle, and wherein the compound is administered in an amount sufficient to result in a mean ratio of compound concentration in the resected brain tissue to compound concentration in plasma of from about 0.50 to about 1.50.

In some embodiments, administering the compound of Formula (I) or a pharmaceutically acceptable salt thereof is over more than one 28 day cycle. In some embodiments, administering the compound of Formula (I) or a pharmaceutically acceptable salt thereof is over two, three, four, five, six, seven, eight, nine, or ten 28 day cycles.

In some embodiments, the compound of Formula (I) or a pharmaceutically acceptable salt thereof is administered in an amount sufficient to result in a mean ratio of compound concentration in the resected brain tissue to compound concentration in plasma of from about 0.50 to about 1.50.

In some embodiments, the glioblastoma is O-6-methylguanine-DNA methyltransferase (MGMT)-positive glioblastoma. In some embodiments, the glioblastoma is O-6-methylguanine-DNA methyltransferase (MGMT)-negative glioblastoma. In some embodiments, the MGMT-positive or MGMT negative glioblastoma is determined by methylation status of the gene, mRNA expression, and/or protein expression. In some embodiments, the glioblastoma has no or a low level O-6-methylguanine-DNA methyltransferase (MGMT) expression.

In some embodiments, the compound of Formula (I), or a pharmaceutically acceptable salt thereof, is adapted for oral administration. In some embodiments, the compound of Formula (I), or a pharmaceutically acceptable salt thereof, is in the form of a tablet, pill, sachet, or capsule of hard of soft gelatin.

In some embodiments, the compound of Formula (I), or a pharmaceutically acceptable salt thereof, has a terminal half-life of at least about 60 hours. In some embodiments, the method provides a platelet count of at least about 100 [*10^{9/}L] in the subject.

In some embodiments, the method provides for a 50% decline from C-C motif chemokine receptor 1 (CCR1) baseline in the subject. In some embodiments, the method provides for a 50% decline from C-C motif chemokine receptor 1 (CCR1) baseline in the subject when measured 74 hours post first dose of the compound.

In some embodiments, the method provides: (a) an AUC (from day 0 to day 28) of at least about 90,000 ng*h/mL; (b) an AUC (from day 0 to day 28) of from about 90,000 ng*h/mL to about 180,000 ng*h/mL; (c) a Cₘₐₓ of at least about 175 ng/mL; (d) a Cₘₐₓ of from about 75 ng/mL to about 1500 ng/mL; and/or (e) a Cₘₐₓ of about 1100 ng/mL.

In some embodiments, (a) the method results in at least about 70% reduction of cancer cell proliferation, tumor cell survival, tumor size, and/or tumor volume; (b) the method results in from about 70% to about 99% reduction of cancer cell proliferation, tumor cell survival, tumor size, and/or tumor volume; (c) the method results in at least about 80% reduction of cancer cell proliferation, tumor cell survival, tumor size, and/or tumor volume; (d) the method results in from about 80% to about 99% reduction of cancer cell proliferation, tumor cell survival, tumor size, and/or tumor volume; and/or (e) the method results in about 70%, about 75%, about 80%, about 85%, about 90%, about 95%, or about 99% reduction of cancer cell proliferation, tumor cell survival, tumor size, and/or tumor volume.

In some embodiments, (a) the method results in at least about 40% reduction of cancer cell proliferation, tumor cell survival, tumor size, and/or tumor volume; (b) the method results in from about 40% to about 99% reduction of cancer cell proliferation, tumor cell survival, tumor size, and/or tumor volume; and/or (c) the method results in about 20%, about 25%, about 30%, about 35%, about 40%, about 45%, about 50%, about 55%, about 60%, about 65%, about 70%, about 75%, about 80%, about 85%, about 90%, about 95%, or about 99% reduction of cancer cell proliferation, tumor cell survival, tumor size, and/or tumor volume.

In some embodiments, (a) the method results in at least about 40% reduction of tumor size and/or tumor volume; (b) the method results in from about 40% to about 99% reduction of tumor size and/or tumor volume; and/or (c) the method results in about 20%, about 25%, about 30%, about 35%, about 40%, about 45%, about 50%, about 55%, about 60%, about 65%, about 70%, about 75%, about 80%, about 85%, about 90%, about 95%, or about 99% reduction of tumor size and/or tumor volume.

In some embodiments, the subject has a reduction of cancer cell proliferation, tumor cell survival, tumor size, and/or tumor volume after administration of the compound, or a pharmaceutically acceptable salt thereof after at least one dose of the compound of Formula (I) or a pharmaceutically acceptable salt thereof and/or after a 28 day cycle.

In some embodiments, the method achieves one or more of the following: (a) a Response Assessment in Neuro-Oncology Criteria (RANO) definition of complete response (CR) in the subject; (b) a Response Assessment in Neuro-Oncology Criteria (RANO) definition of partial response (PR) in the subject; and (c) a Response Assessment in Neuro-Oncology Criteria (RANO) definition of stable disease (SD) in the subject.

In a further embodiment, the disclosure encompasses a combination of: (a) a pharmaceutical composition comprising a compound having the structure of Formula (I),

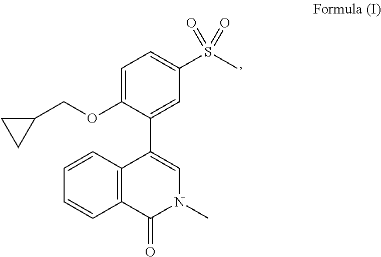 [SMILES: Cn1cc(-c2cc(S(C)(=O)=O)ccc2OCC2CC2)c2ccccc2c1=O]

or a pharmaceutically acceptable salt thereof, (b) temozolomide, and (c) radiotherapy, for use in treating glioblastoma multiforme (GBM) in a newly diagnosed GBM subject in need thereof, wherein: (i) radiotherapy, temozolomide, and the composition comprising the compound of Formula (I), or a pharmaceutically acceptable salt thereof, are formulated for concomitant administration to the newly diagnosed GBM subject; thereafter (ii) temozolomide and the compound of Formula (I), or a pharmaceutically acceptable salt thereof, are formulated for adjunctive administeration without radiotherapy; and thereafter (iii) the composition comprising the compound of Formula (I), or a pharmaceutically acceptable salt thereof, is formulated for administration as a monotherapy.

In another embodiment, the disclosure encompasses a combination of: (a) a pharmaceutical composition comprising a compound having the structure of Formula (I),

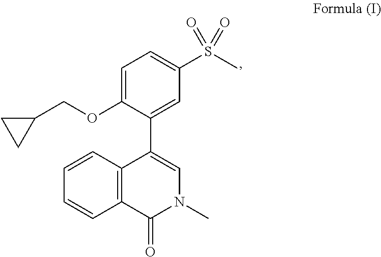 [SMILES: Cn1cc(-c2cc(S(C)(=O)=O)ccc2OCC2CC2)c2ccccc2c1=O]

or a pharmaceutically acceptable salt thereof, and (b) temozolomide, for use in treating glioblastoma multiforme (GBM) in a subject in need thereof, wherein the temozolomide and the composition comprising a compound of Formula (I), or a pharmaceutically acceptable salt thereof, are formulated for adjunctive administeration without radiotherapy, and wherein the temozolomide and the composition comprising the compound of Formula (I), or a pharmaceutically acceptable salt thereof, are formulated for administration in a 28 day cycle.

In another embodiment, the disclosure encompasses a composition comprising a compound having the structure of Formula (I),

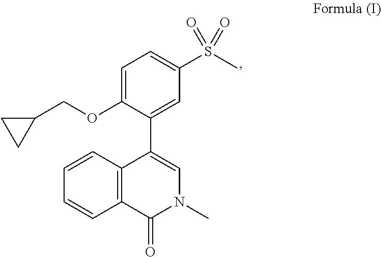 [SMILES: Cn1cc(-c2cc(S(C)(=O)=O)ccc2OCC2CC2)c2ccccc2c1=O]

or a pharmaceutically acceptable salt thereof, for use in treating glioblastoma multiforme (GBM) in a subject in need thereof, wherein the composition comprising the compound of Formula (I), or a pharmaceutically acceptable salt thereof, is formulated for administeration as a monotherapy treatment of 45 mg on Days 1-4 of a 28 day cycle, and wherein the composition comprising the compound of Formula (I), or a pharmaceutically acceptable salt thereof, is formulated for administeration in an amount sufficient to result in a mean ratio of compound concentration in the resected brain tissue to compound concentration in plasma of from about 0.50 to about 1.50.

For all of the combinations and compositions described herein, the glioblastoma can be O-6-methylguanine-DNA methyltransferase (MGMT)-positive glioblastoma, or the glioblastoma can be O-6-methylguanine-DNA methyltransferase (MGMT)-negative glioblastoma. In addition, the MGMT-positive or MGMT negative glioblastoma can be determined by methylation status of the gene, mRNA expression, and/or protein expression.

For all of the combinations and compositions described herein, the glioblastoma can have no or a low level O-6-methylguanine-DNA methyltransferase (MGMT) expression.

In a further aspect for all of the combinations and compositions described herein: (a) the compound of Formula (I), or a pharmaceutically acceptable salt thereof, is adapted for oral administration; and/or (b) the compound of Formula (I), or a pharmaceutically acceptable salt thereof, is in the form of a tablet, pill, sachet, or capsule of hard of soft gelatin. In addition, the compound of Formula (I), or a pharmaceutically acceptable salt thereof, can have a terminal half-life of at least about 60 hours; and/or upon administration to a subject the combination can provide a platelet count of at least about 100 [*10⁹L] in the subject; and/or upon administration to a subject the combination or composition can provide for a 50% decline from C-C motif chemokine receptor 1 (CCR1) baseline in the subject; and/or upon administration to a subject the combination or composition can provide for a 50% decline from C-C motif chemokine receptor 1 (CCR1) baseline in the subject when measured 74 hours post first dose of the compound; and/or upon administration to a subject the combination or composition can provide: (i) an AUC (from day 0 to day 28) of at least about 90,000 ng*h/mL; (ii) an AUC (from day 0 to day 28) of from about 90,000 ng*h/mL to about 180,000 ng*h/mL; (iii) a Cₘₐₓ of at least about 175 ng/mL; (iv) a Cₘₐₓ of from about 75 ng/mL to about 1500 ng/mL; and/or (v) a Cₘₐₓ of about 1100 ng/mL.

In a further aspect for all of the combinations and compositions described herein, upon administration to a subject the combination or composition provides: (a) at least about 70% reduction of cancer cell proliferation, tumor cell survival, tumor size, and/or tumor volume; (b) about 70% to about 99% reduction of cancer cell proliferation, tumor cell survival, tumor size, and/or tumor volume; (c) at least about 80% reduction of cancer cell proliferation, tumor cell survival, tumor size, and/or tumor volume; (d) from about 80% to about 99% reduction of cancer cell proliferation, tumor cell survival, tumor size, and/or tumor volume; (e) about 70%, about 75%, about 80%, about 85%, about 90%, about 95%, or about 99% reduction of cancer cell proliferation, tumor cell survival, tumor size, and/or tumor volume; (f) at least about 40% reduction of cancer cell proliferation, tumor cell survival, tumor size, and/or tumor volume; (g) from about 40% to about 99% reduction of cancer cell proliferation, tumor cell survival, tumor size, and/or tumor volume; and/or (h) about 20%, about 25%, about 30%, about 35%, about 40%, about 45%, about 50%, about 55%, about 60%, about 65%, about 70%, about 75%, about 80%, about 85%, about 90%, about 95%, or about 99% reduction of cancer cell proliferation, tumor cell survival, tumor size, and/or tumor volume.

In a further aspect for all of the combinations and compositions described herein, (a) the combination results in at least about 40% reduction of tumor size and/or tumor volume; (b) the combination results in from about 40% to about 99% reduction of tumor size and/or tumor volume; and/or (c) the combination results in about 20%, about 25%, about 30%, about 35%, about 40%, about 45%, about 50%, about 55%, about 60%, about 65%, about 70%, about 75%, about 80%, about 85%, about 90%, about 95%, or about 99% reduction of tumor size and/or tumor volume.

In a further aspect for all of the combinations and compositions described herein, upon administration the subject has a reduction of cancer cell proliferation, tumor cell survival, tumor size, and/or tumor volume after administration of the compound, or a pharmaceutically acceptable salt thereof after at least one dose of the compound of Formula (I) or a pharmaceutically acceptable salt thereof and/or after a 28 day cycle.

In a further aspect for all of the combinations and compositions described herein, the combination or composition achieves one or more of the following: (a) a Response Assessment in Neuro-Oncology Criteria (RANO) definition of complete response (CR) in the subject; (b) a Response Assessment in Neuro-Oncology Criteria (RANO) definition of partial response (PR) in the subject; and (c) a Response Assessment in Neuro-Oncology Criteria (RANO) definition of stable disease (SD) in the subject.

Both the foregoing summary and the following description of the drawings and detailed description are exemplary and explanatory. They are intended to provide further details of the invention, but are not to be construed as limiting. Other objects, advantages, and novel features will be readily apparent to those skilled in the art from the following detailed description of the invention.

### BRIEF DESCRIPTION OF THE DRAWINGS

FIG. 1 shows the CCR1% at 74 hours and IDH mutation relationship versus Days on Study for Example 1.

FIG. 2 shows the time on study versus IDH status for the subjects treated in Example 1.

FIG. 3 shows the CCR1% at 74 hours and MGMT methylation status versus to time on treatment for Example 1.

FIG. 4 shows the HEXIM1% at the time of biopsy versus plasma concentration in Example 1.

FIG. 5 shows the CCR1% versus time after the first dose in Example 1 at 30 mg of Compound A in CC-90010 GBM-001.

FIG. 6 shows the CCR1% versus time after the first dose in Example 2 at 30 mg of Compound A CC-90010 GBM-002.

FIG. 7 shows the adjuvant dosing and concomitant dosing schedules for Compound A.

FIG. 8 shows the adjuvant dosing and concomitant dosing schedules for Compound A in Part B of the study.

### DETAILED DESCRIPTION

### I. Overview

The present invention is directed to methods of treating glioblastoma with a therapeutically effective amount of a bromodomain inhibitor having a structure of Formula (I), or a pharmaceutically acceptable salt thereof.

In some embodiments, the glioblastoma is O-6-methylguanine-DNA methyltransferase (MGMT)-positive glioblastoma. In some embodiments, the glioblastoma is O-6-methylguanine-DNA methyltransferase (MGMT)-negative glioblastoma. The MGMT status may be determined by methylation status of the gene, mRNA expression, and/or protein expression. In some embodiments, the MGMT-positive or MGMT negative glioblastoma is determined by methylation status of the gene, mRNA expression, and/or protein expression.

The methods described herein include using a therapeutically effective amount of a bromodomain inhibitor having a structure of Formula (I) in a concomitant therapy, an adjuvant therapy and as a monotherapy. Use as a concomitant therapy includes concomitant use with a chemotherapy drug, such as temozolomide, and radiation therapy. Use as an adjuvant therapy includes use with a chemotherapy drug, such as temozolomide, but no radiation therapy. Use as a monotherapy includes use of the bromodomain inhibitor having a structure of Formula (I) with any other medicament or radiation therapy.

The methods described herein include using a therapeutically effective amount of a bromodomain inhibitor having a structure of Formula (I) as a monotherapy, and using a bromodomain inhibitor in combination with a chemotherapy drug, such as temozolomide, and using a bromodomain inhibitor in combination with a chemotherapy drug and radiation therapy.

Substituted heterocyclic derivative compounds useful as bromodomain inhibitors include isoquinolinones and related heterocyclic structures that are typically substituted at the 4-position with an aryl, a heteroaryl or a similar group, and at the nitrogen atom of the isoquinolinone or related heterocyclic structure with a small alkyl group (e.g., methyl group). Examples of such compounds are disclosed in U.S. patent application Ser. No. 14/517,705 (U.S. Pat. No. 9,034,900).

In any of the embodiments described herein, the bromodomain inhibitor compound is trotabresib, 4-[2-(cyclopropylmethoxy)-5-methylsulfonylphenyl]-2-methylisoquinolin-1-one, which has the following structure of Formula (I):

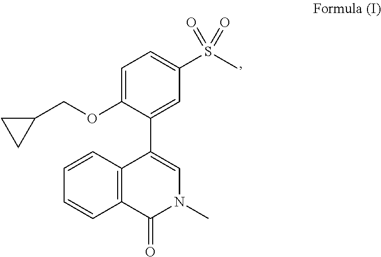 [SMILES: Cn1cc(-c2cc(S(C)(=O)=O)ccc2OCC2CC2)c2ccccc2c1=O]

or the pharmaceutically acceptable salt thereof (herein referred to as “Compound A”). The above compound has the chemical formula of C₂₁H₂₁NO₄S and a molecular weight of 383.46. The synthesis of this compound is disclosed in in U.S. patent application Ser. No. 14/517,705 (U.S. Pat. No. 9,034,900).

Pharmaceutically acceptable salts of include, but are not limited to, acid addition salts, formed by reacting the compound with a pharmaceutically acceptable inorganic acid, such as, for example acid addition salts, formed by reacting the compound with a pharmaceutically acceptable inorganic acid, such as, for example, hydrochloric acid, hydrobromic acid, sulfuric acid, phosphoric acid, metaphosphoric acid, and the like; or with an organic acid, such as, for example, acetic acid, propionic acid, hexanoic acid, cyclopentanepropionic acid, glycolic acid, pyruvic acid, lactic acid, malonic acid, succinic acid, malic acid, maleic acid, fumaric acid, trifluoroacetic acid, tartaric acid, citric acid, benzoic acid, cinnamic acid, mandelic acid, methanesulfonic acid, ethanesulfonic acid, 1,2-ethanedisulfonic acid, 2-hydroxyethanesulfonic acid, benzenesulfonic acid, toluenesulfonic acid, 2-naphthalenesulfonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, butyric acid, phenylacetic acid, phenylbutyric acid, valproic acid, and the like.

When discussing the dosage amounts of the present invention, the invention refers to the active moiety consistent with guidance from the USP Salt Policy. The USP Salt Policy is a naming and labeling policy applicable to drug products that contain an active ingredient that is a salt. The USP Salt policy stipulates that USP will use the name of the active moiety, instead of the name of the salt. The dosage amounts, e.g., the miligram amounts, referred to herein thus reference the active moiety of Compound A, and not the milligram amounts of a pharmaceutically acceptable salt of Compound A.

In any of the methods described herein, the single dose of the compound, or a pharmaceutically acceptable salt thereof, is about 15 mg. In some embodiments, the single dose of the compound, or a pharmaceutically acceptable salt thereof, is about 15 mg, about 20 mg, about 25 mg, about 30 mg, about 35 mg, about 40 mg, about 45 mg, about 50 mg, or about 55 mg. In some embodiments, the single dose of the compound, or a pharmaceutically acceptable salt thereof, is from about 15 mg to about 55 mg, from about 20 mg to about 50 mg, or from about 30 mg to about 50 mg. In some embodiments, the single dose of the compound, or a pharmaceutically acceptable salt thereof, is about 15 mg, about 20 mg, about 25 mg, about 30 mg, about 35 mg, about 40 mg, about 45 mg, about 50 mg, or about 55 mg. In some embodiments, the single dose of the compound, or a pharmaceutically acceptable salt thereof, is about 35 mg. In some embodiments, the single dose of the compound, or a pharmaceutically acceptable salt thereof, is about 45 mg.

In some embodiments, temozolomide (TMZ) is used in combination with the bromodomain inhibitors described herein. Temozolomide is imidazotetrazine derivative having the chemical name 3,4-dihydro-3-methyl-4-oxoimidazo[5,1-d]-as-tetrazine-8-carboxamide. Temozolomide has a molecular formula of C₆H₆N₆O₂, and a molecular weight of 194.15. The structural formula is:

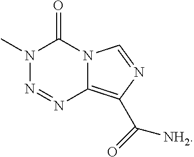

Temozolomide is commercially available as an injectable form administered via intravenous infusion. Temozolomide, sold as TEMODAR® by Merck & Co., Inc. is administered at an initial dose of 150 mg/m² intravenously once daily for 5 consecutive days per 28-day treatment cycle. The dose may be increased to 200 mg/m²/day for 5 consecutive days per 28-day treatment cycle.

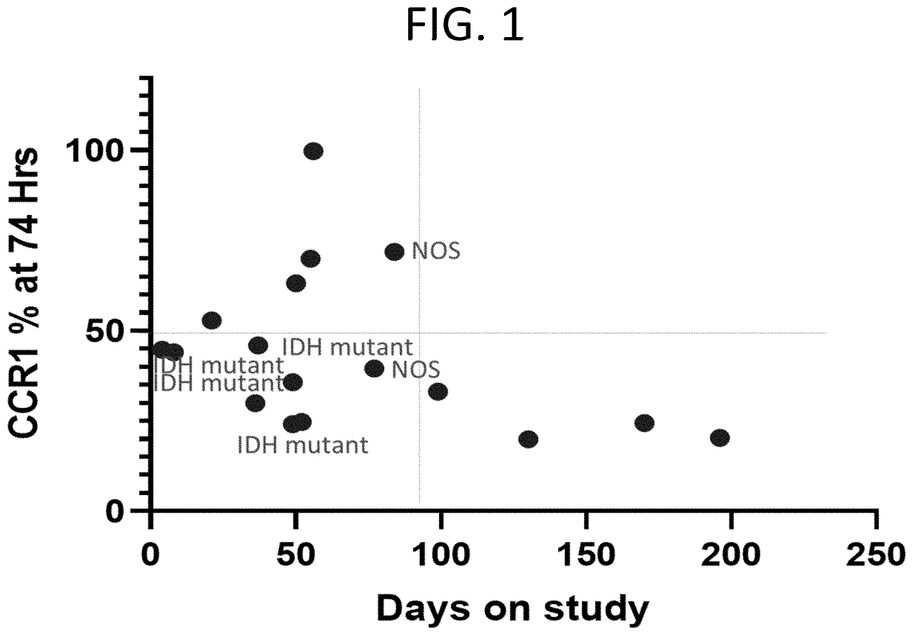

Experimental Results: As detailed in the examples below, Compound A (also known as CC-90010, trotabresib, and to 4-[2-(cyclopropylmethoxy)-5-methylsulfonylphenyl]-2-methylisoquinolin-1-one) was found to be useful in certain clinical protocols for treating glioblastoma multiforme (GBM). In particular, Example 1 describes treating patients with progressive or recurrent astrocytoma (1 patient) or recurrent glioblastoma (19 patients) who have failed radiation and chemotherapy. The Example details that Compound A showed good tumor tissue penetration, with pharmacodynamic signals of response, and was well tolerated. In addition, Compound A was found to have high brain penetrance. Brain tumor tissue concentrations of Compound A are lower than in plasma, with a tissue:plasma ratio of 0.14. The results are detailed in FIGS. 1-6.

Brain tumor tissue penetration data for various small molecule targeted therapies shows marked variation in relative tissue concentrations across agents, with tissue:plasma ratios of median 0.71 for imatinib, mean 0.25 for olaparib, and 0.06-0.08 for erlotinib. In contrast, both lapatinib and gefitinib show accumulation in glioblastoma tumor tissue, with a mean tissue:plasma ratio of 13.0 for lapatinib and median gefitinib concentrations in tumor tissue and plasma of 4100 ng/g and 181 ng/mL, respectively (P<0.0001). However, neither lapatinib nor gefitinib have shown evidence of antitumor activity in glioblastoma despite the high drug concentrations reported in tumor tissue.

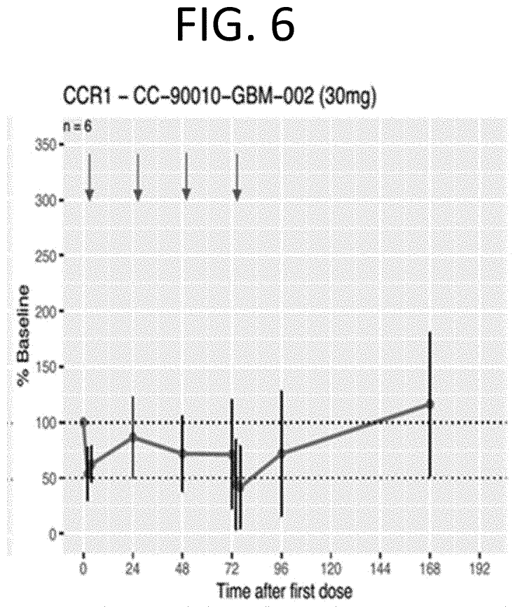

Example 2 details the results of a study combining Compound A with temozolomide (TMZ), with or without radiotherapy (RT), in treating newly diagnosed glioblastoma patients. In this study, Compound A was administered both as an adjuvant therapy to TMZ, and also as a monotherapy. Specifically, the treatment groups were concomitant therapy (Compound A+RT+TMZ followed by adjuvant therapy and then Compound A monotherapy) and adjuvant therapy (Compound A+TMZ followed Compound A monotherapy). See also FIGS. 6-8. There were no treatment-related deaths. In sum, adjuvant Compound A plus TMZ and concomitant Compound A plus TMZ and RT in patients with newly diagnosed GBM continued to be well tolerated in long-term follow-up with no new safety signals. The safety profile remained consistent with previous studies of Compound A monotherapy and adjuvant TMZ. As of the study data cutoff date, median total duration of treatment was 37 weeks and 46 weeks in the adjuvant and concomitant cohorts, respectively. Five (28%) and 4 (29%) patients in the adjuvant and concomitant cohorts, respectively, remained on treatment. Six-month PFS rates were 57.8% and 69.2% in the adjuvant and concomitant cohorts, respectively. Compound A plasma PK in part B were similar to those observed in clinical trials investigating Compound A monotherapy. Coadministration with RT and TMZ did not appear to impact Compound A PK.

### II. Pharmaceutical Compositions

In any of the embodiments described herein, the bromodomain inhibitors described herein, or the pharmaceutically acceptable salts thereof, are formulated into pharmaceutical compositions.

Pharmaceutical compositions are formulated in a conventional manner using one or more pharmaceutically acceptable inactive ingredients that facilitate processing of the active compounds into preparations that are used pharmaceutically. Proper formulation is dependent upon the route of administration chosen. In some embodiments, a pharmaceutical composition disclosed herein comprises one or more pharmaceutically acceptable carriers, such as an aqueous carrier, buffer, and/or diluent.

The compounds described herein may be combined or coordinately administered with a suitable carrier or vehicle depending on the route of administration. As used herein, the term “carrier” means a pharmaceutically acceptable solid or liquid filler, diluent or encapsulating material. A water-containing liquid carrier can comprise pharmaceutically acceptable additives such as acidifying agents, alkalizing agents, antimicrobial preservatives, antioxidants, buffering agents, chelating agents, complexing agents, solubilizing agents, humectants, solvents, suspending and/or viscosity-increasing agents, tonicity agents, wetting agents or other biocompatible materials. A tabulation of ingredients listed by the above categories can be found in the U.S. Pharmacopeia National Formulary, 1857-1859, and (1990). Some examples of the materials which can serve as pharmaceutically acceptable carriers are sugars, such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil, safflower oil, sesame oil, olive oil, corn oil and soybean oil; glycols, such as propylene glycol; polyols such as glycerin, sorbitol, mannitol and polyethylene glycol; esters such as ethyl oleate and ethyl laurate; agar; buffering agents such as magnesium hydroxide and aluminum hydroxide; alginic acid; pyrogen free water; isotonic saline; Ringer's solution, ethyl alcohol and phosphate buffer solutions, as well as other nontoxic compatible substances used in pharmaceutical formulations. Wetting agents, emulsifiers and lubricants such as sodium lauryl sulfate and magnesium stearate, as well as coloring agents, release agents, coating agents, sweetening, flavoring and perfuming agents, preservatives and antioxidants can also be present in the compositions, according to the desires of the formulator. Examples of pharmaceutically acceptable antioxidants include water soluble antioxidants such as ascorbic acid, cysteine hydrochloride, sodium bisulfite, sodium metabisulfite, sodium sulfite and the like; oil-soluble antioxidants such as ascorbyl palmitate, butylated hydroxyanisole (BHA), butylated hydroxytoluene (BHT), lecithin, propyl gallate, alpha-tocopherol and the like; and metal-chelating agents such as citric acid, ethylenediamine tetraacetic acid (EDTA), sorbitol, tartaric acid, phosphoric acid and the like.

Pharmaceutical compositions according to the invention may also comprise one or more binding agents, filling agents, lubricating agents, suspending agents, sweeteners, flavoring agents, preservatives, buffers, wetting agents, disintegrants, effervescent agents, and other excipients. Such excipients are known in the art. Examples of filling agents include lactose monohydrate, lactose anhydrous, and various starches; examples of binding agents include various celluloses and cross-linked polyvinylpyrrolidone, microcrystalline cellulose, such as Avicel® PH101 and Avicel® PH102, microcrystalline cellulose, and silicified microcrystalline cellulose (ProSolv SMCC™). Suitable lubricants, including agents that act on the flowability of the powder to be compressed, may include colloidal silicon dioxide, such as Aerosil® 200, talc, stearic acid, magnesium stearate, calcium stearate, and silica gel. Examples of sweeteners may include any natural or artificial sweetener, such as sucrose, xylitol, sodium saccharin, cyclamate, aspartame, and acesulfame. Examples of flavoring agents are Magnasweet® (trademark of MAFCO), bubble gum flavor, and fruit flavors, and the like. Examples of preservatives include potassium sorbate, methylparaben, propylparaben, benzoic acid and its salts, other esters of parahydroxybenzoic acid such as butylparaben, alcohols such as ethyl or benzyl alcohol, phenolic compounds such as phenol, or quaternary compounds such as benzalkonium chloride.

Any pharmaceutical used for therapeutic administration can be sterile. Sterility is readily accomplished by for example filtration through sterile filtration membranes (e.g., 0.2 micron membranes). Any pharmaceutically acceptable sterility method can be used in the compositions of the invention.

The pharmaceutical composition comprising the compounds described herein, or the salts thereof, will be formulated and dosed in a fashion consistent with good medical practice, taking into account the clinical condition of the individual patient, the method of administration, the scheduling of administration, and other factors known to practitioners.

### III. Methods of Treatment

Glioma is the most common malignant tumor of the central nervous system (CNS). Therapeutic efficacy of glioma treatment is greatly limited by the blood-brain barrier (BBB) and blood-brain tumor barrier (BBTB), which restrict the passage of most drugs into the brain and tumors, and thus limits the therapeutic efficacy of most chemotherapy drugs. To have sufficient concentration of drug passing through the BBB and into the brain and brain tumor, there needs to be a sufficient concentration of unbound circulating drug in the plasma.

The present application provides methods of treating glioblastoma using any one of the bromodomain inhibitors described herein, or pharmaceutically acceptable salts thereof. In one embodiment, the bromodomain inhibitor is trotabresib, referred herein as Compound A.

In some embodiments, the mean ratio of compound concentration (e.g., Compound A concentration) in the resected brain tissue to compound concentration (e.g., Compound A concentration) in blood plasma is from about 0.50 to about 1.50, including from about 0.59 to about 0.10, from about 0.65 to about 0.95, from about 0.70 to about 0.95, and from about 0.75 to about 0.95. In some embodiments, the mean ratio of Compound A concentration in the resected brain tissue to Compound A concentration in blood plasma is from about 0.59 to about 1.10, such as about 0.84.

One solution for achieving the above mean ratio is by adjusting the dosage of the compound having the structure of Formula (I), Compound A. However, administrating large dosage amounts of Compound A to obtain circulating drug in blood plasma may result in levels that are toxic and/or have many adverse events (AEs), such as platelet nadirs (low points) that are too low.

Another solution is to reduce the dosage and to adjust the dosing frequency. For instance, prolonged exposure, e.g. a Cₘᵢₙ plasma concentration of a drug, such as Compound A, which maintains blood levels where AEs are manageable, is insufficient to push the drug across BBB and have a therapeutic outcome.

For Compound A, the inventors surprisingly found a therapeutic regimen comprising a combination of a maximum tolerated dose in combination with a unique dosing regimen as disclosed herein. In particular, the dosing regimens described herein for Compound A maximize the blood plasma accumulation of Compound A for BBB penetration, and thus achieves the desired brain penetrance. Furthermore, the dosing regimens for Compound A when concomitantly administered with temozolomide maximizes MGMT depletion. Furthermore, the dosing regimens disclosed herein also achieved the desired plasma concentration while balancing the AEs, such as platelet nadirs. The clinical data described in the Examples demonstrate that the dosing regimens and resulting blood brain penetrance levels provide a surprising and unexpected significant clinical effect.

In any of the methods described herein, the AUC (from day 0 to day 28) is at least about 90,000 ng*h/mL. In some embodiments, AUC (from day 0 to day 28) is about 90,000 ng*h/mL to about 180,000 ng*h/mL, including about 90,000 ng*h/mL, about 100,000 ng*h/mL, about 110,000 ng*h/mL, about 120,000 ng*h/mL, about 130,000 ng*h/mL, about 140,000 ng*h/mL, about 150,000 ng*h/mL, about 160,000 ng*h/mL, about 170,000 ng*h/mL, and about 180,000 ng*h/mL.

In any of the methods described herein, the Cₘₐₓ is at least about 175 ng/mL. In some embodiments, the Cₘₐₓ is about 75 ng/mL to about 1500 ng/mL, including about 75 ng/mL, about 80 ng/mL, about 85 ng/mL, about 90 ng/mL, about 95 ng/mL, about 100 ng/mL, about 200 ng/mL, about 300 ng/mL, about 400 ng/mL, about 500 ng/mL, about 600 ng/mL, about 700 ng/mL, about 800 ng/mL, about 900 ng/mL, about 1000 ng/mL, about 1100 ng/mL, about 1200 ng/mL, about 1300 ng/mL, about 1400 ng/mL, and about 1500 ng/mL. In some embodiments, the Cₘₐₓ is about 1100 ng/mL.

In some embodiments, the methods described herein provide a platelet count of at least about 100 [*10⁹′L]. In some embodiments, the methods described herein provide at least about a 50%, at least about a 55%, at least about a 60%, at least about a 65%, at least about a 70%, at least about a 75%, at least about a 80% or at least about a 85% decline from C-C motif chemokine receptor 1 (CCR1) baseline. In some embodiments, the methods described herein provide at least about a 50% decline from C-C motif chemokine receptor 1 (CCR1) baseline. As shown by the Examples, at least about a 50% decline from CCR1 baseline is observed following the first and last dose of the bromodomain inhibitor compound, such as Compound A, with the deepest suppression following the last dose of the bromodomain compound in the first cycle. In some embodiments, the methods provide at least about a 50% decline from C-C motif chemokine receptor 1 (CCR1) baseline at about 74 hours.

According to an embodiment of the present invention, Compound A is administered as a monotherapy. In this embodiment, 45 mg of Compound A is administered on Days 1-4 of a 28-day treatment cycle. The treatment cycle is repeated as appropriate, such as about 2 to 24 times, 2-18 times, 2-12 times, etc. until disease progression.

According to another embodiment of the present invention, Compound A is administered as an adjuvant therapy in combination with temozolomide without radiation therapy to treat GBM. According to this embodiment, 15 mg, 30 mg, or 45 mg of Compound A is on Days 1-4, and 150 mg/m² temozolomide is administered on Days 1-5 of a 28-day cycle. The treatment cycle (e.g., 28-day cycle) is repeated as appropriate, such as about 2 to 24 times, 2 to 18 times, 2 to 12 times, 2 to 10 times, 2 to 8 times, 2 to 6 times, or 2 to 4 times. Preferably, the treatment (e.g., 28-day cycle) is repeated for 6 cycles. For cycles 2 on, Compound A is continued at 4 days on, 24 days off to complete a cycle. Temozolomide, however, is given at 200 mg/m² once daily on Days 1-5 of the 28 day cycle.

According to yet a further embodiment of the present invention, Compound A and temozolomide are administered with concomitant radiation therapy. According to this embodiment, 15 mg or 30 mg of Compound A is administered on Days 1-4 of week 1 and 5, 75 mg/m² of temozolomide is administered once daily for 42 days, and with the patient undergoing daily radiation therapy.

Provided in one aspect is a method of treating glioblastoma multiforme (GBM) in a newly diagnosed GBM subject in need thereof comprising: (i) administering to the newly diagnosed GBM subject, a concomitant treatment of radiotherapy with temozolomide and the compound having the structure of Formula (I),

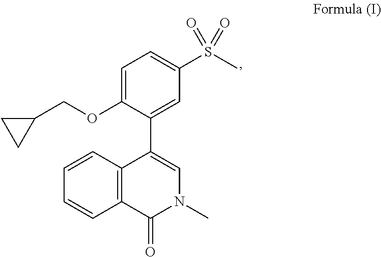 [SMILES: Cn1cc(-c2cc(S(C)(=O)=O)ccc2OCC2CC2)c2ccccc2c1=O]

or a pharmaceutically acceptable salt thereof; thereafter

(ii) adjunctively administering temozolomide and the compound of Formula (I) or a pharmaceutically acceptable salt thereof without radiotherapy; and thereafter

(iii) administering the compound of Formula (I) or a pharmaceutically acceptable salt thereof as a monotherapy.

In some embodiments, step (i) comprises a 42-day treatment regimen comprising administering the compound of Formula (I) or a pharmaceutically acceptable salt thereof on Days 1-4 and Days 35-39. In some embodiments, step (i) comprises a 42-day treatment regimen comprising administering 15 mg or 30 mg of the compound of Formula (I) or a pharmaceutically acceptable salt on Days 1-4 and Days 35-39. In some embodiments, step (i) comprises a 42-day treatment regimen comprising administering temozolomide once daily for the 42 days. In some embodiments, step (i) comprises a 42-day treatment regimen comprising administering 75 mg/m² of temozolomide once daily for the 42 days.

In some embodiments, step (ii) comprises a 28-day cycle comprising administering the compound of Formula (I) or a pharmaceutically acceptable salt thereof on Days 1-4. In some embodiments, step (ii) comprises a 28-day cycle comprising administering 15 mg, 30 mg, or 45 mg of compound of Formula (I) or a pharmaceutically acceptable salt thereof on Days 1-4. In some embodiments, step (ii) comprises a 28-day cycle comprising administering temozolomide on Days 1-5. In some embodiments, step (ii) comprises a 28-day cycle comprising administering 150 mg/m² of temozolomide on Days 1-4. In some embodiments, step (ii) comprises a 28 day cycle repeating from 2 to 6 times, and wherein 15 mg, 30 mg, or 45 mg of the compound of Formula (I) or a pharmaceutically acceptable salt is administered on Days 1-4 of cycles 2-6 and wherein temozolomide is administered at a dose of 200 mg/m² on Days 1-5 of cycles 2-6.

In some embodiments, step (iii) comprises a 28-day cycle comprising administering the compound of Formula (I) or a pharmaceutically acceptable salt thereof on Days 1-4. In some embodiments, step (iii) comprises a 28-day cycle comprising administering 45 mg of compound of Formula (I) or a pharmaceutically acceptable salt thereof on Days 1-4. In some embodiments, step (iii) comprises a 28-day cycle comprising administering the compound of Formula (I) or a pharmaceutically acceptable salt thereof is over one, two, three, four, five, six, seven, eight, nine, or ten cycles.

Provided in another aspect is a method of treating glioblastoma multiforme (GBM) in a subject in need thereof comprising adjunctively administering temozolomide and the compound of having the structure of Formula (I),

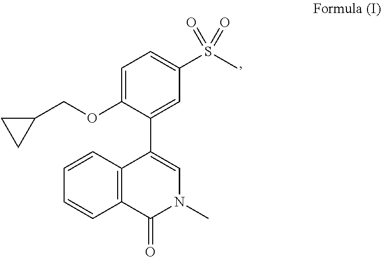 [SMILES: Cn1cc(-c2cc(S(C)(=O)=O)ccc2OCC2CC2)c2ccccc2c1=O]

or a pharmaceutically acceptable salt thereof without radiotherapy, and wherein administering the temozolomide and the compound of Formula (I) are in a 28 day cycle.

In some embodiments, administering the compound of Formula (I) or a pharmaceutically acceptable salt thereof is on Days 1-4. In some embodiments, 15 mg, 30 mg, or 45 mg of the compound of Formula (I) or a pharmaceutically acceptable salt is administered on Days 1-4. In some embodiments, administering temozolomide is on Days 1-5. In some embodiments, 150 mg/m² of temozolomide is administered on Days 1-4 of a 28-day cycle. In some embodiments, the 28-day cycle repeats 2 to 6 times, and wherein 15 mg, 30 mg, or 45 mg of the compound of Formula (I) or a pharmaceutically acceptable salt is administered on Days 1-4 of cycles 2-6, and wherein temozolomide is administered at a dose of 200 mg/m² on Days 1-5 of cycles 2-6.

Provided in one aspect is a method of treating glioblastoma multiforme (GBM) in a subject in need thereof comprising administering a monotherapy treatment of 45 mg of a compound having the structure of Formula (I),

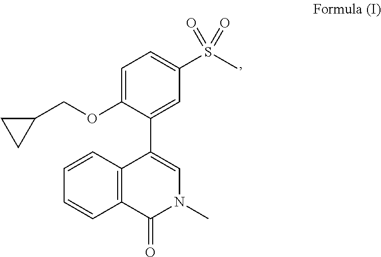 [SMILES: Cn1cc(-c2cc(S(C)(=O)=O)ccc2OCC2CC2)c2ccccc2c1=O]

or a pharmaceutically acceptable salt thereof on Days 1-4 of a 28 day cycle, and
wherein the compound is administered in an amount sufficient to result in a mean ratio of compound concentration in the resected brain tissue to compound concentration in plasma of from about 0.50 to about 1.50.

In some embodiments, administering the compound of Formula (I) or a pharmaceutically acceptable salt thereof is over more than one 28 day cycle. In some embodiments, administering the compound of Formula (I) or a pharmaceutically acceptable salt thereof is over two, three, four, five, six, seven, eight, nine, or ten 28 day cycles.

In any embodiment described herein, the glioblastoma is O-6-methylguanine-DNA methyltransferase (MGMT)-positive glioblastoma. In any embodiment, the glioblastoma is O-6-methylguanine-DNA methyltransferase (MGMT)-negative glioblastoma. In any embodiment, the MGMT-positive or MGMT negative glioblastoma is determined by methylation status of the gene, mRNA expression, and/or protein expression. In any embodiment, the glioblastoma has no or low level O-6-methylguanine-DNA methyltransferase (MGMT) expression.

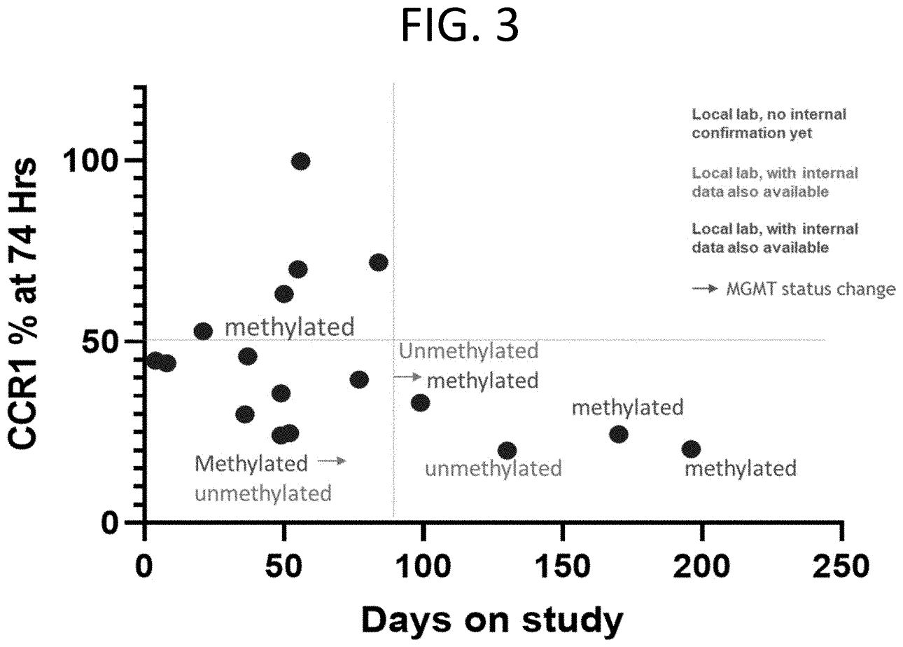

A “low level expression” can be, for example, less than about 100, less than about 95, less than about 90, less than about 85, less than about 80, less than about 75, less than about 70, less than about 65, less than about 60, less than about 55, less than about 50, less than about 45, less than about 40, less than about 35, less than about 30, less than about 25, less than about 20, less than about 15, less than about 10, or less than about 5 mol/mg protein, See Ishiguro et al., J. Cancer Ther., 4(4):919-931 (2013), at FIG. 3 (showing a comparison of MGMT expression in normal and malignant tissue).

In some embodiments, the glioblastoma is MGMT-negative glioblastoma. In some embodiments, the MGMT negative glioblastoma is determined by methylation status of the gene, mRNA expression, and/or protein expression.

In some embodiments, the compound, or a pharmaceutically acceptable salt thereof, has a terminal half-life of at least about 50 hours, at least about 55 hours, at least about 60 hours, at least about 65 hours, or at least about 70 hours. In some embodiments, the compound, or a pharmaceutically acceptable salt thereof, has a terminal half-life of at least about 60 hours.

In some embodiments, the method describes herein provides a platelet count of at least about 100 [*10⁹/L] in the subject. In some embodiments, the method describes herein provides for a 50%, 55%, 60%, 65%, 70%, 75%, 80% or 85% decline from C-C motif chemokine receptor 1 (CCR1) baseline in the subject. In some embodiments, the method describes herein provides for a 50% decline from C-C motif chemokine receptor 1 (CCR1) baseline in the subject. As shown by the Examples, a 50% decline from CCR1 baseline is observed following the first and last dose of the bromodomain inhibitor compound, such as Compound A with the deepest suppression following the last dose of the bromodomain compound in the first cycle. In some embodiments, the method provides for a 50% decline from C-C motif chemokine receptor 1 (CCR1) baseline in the subject when measured at 74 hours.

In any of the embodiments described herein, the method results in at least about 70% reduction of cancer cell proliferation, tumor cell survival, tumor size, and/or tumor volume. In some embodiments, the method results in from about 70% to about 99% reduction of cancer cell proliferation, tumor cell survival, tumor size, and/or tumor volume. In some embodiments, the method results in at least about 80% reduction of cancer cell proliferation, tumor cell survival, tumor size, and/or tumor volume. In some embodiments, the method results in from about 80% to about 99% reduction of cancer cell proliferation, tumor cell survival, tumor size, and/or tumor volume. In some embodiments, the method results in about 70%, about 75%, about 80%, about 85%, about 90%, about 95%, or about 99% reduction of cancer cell proliferation, tumor cell survival, tumor size, and/or tumor volume.

In any of the embodiments described herein, the method results in at least about 70% reduction of cancer cell proliferation. In some embodiments, the method results in from about 70% to about 99% reduction of cancer cell proliferation. In some embodiments, the method results in at least about 80% reduction of cancer cell proliferation. In some embodiments, the method results in from about 80% to about 99% reduction of cancer cell proliferation. In some embodiments, the method results in about 70%, about 75%, about 80%, about 85%, about 90%, about 95%, or about 99% reduction of cancer cell proliferation.

In any of the embodiments described herein, the method results in at least about 70% reduction of tumor cell survival. In some embodiments, the method results in from about 70% to about 99% reduction of tumor cell survival. In some embodiments, the method results in at least about 80% reduction of tumor cell survival. In some embodiments, the method results in from about 80% to about 99% reduction of tumor cell survival. In some embodiments, the method results in about 70%, about 75%, about 80%, about 85%, about 90%, about 95%, or about 99% reduction of tumor cell survival.

In any of the embodiments described herein, the method results in at least about 70% reduction of tumor size. In some embodiments, the method results in from about 70% to about 99% reduction of tumor size. In some embodiments, the method results in at least about 80% reduction of tumor size. In some embodiments, the method results in from about 80% to about 99% reduction of tumor size. In some embodiments, the method results in about 70%, about 75%, about 80%, about 85%, about 90%, about 95%, or about 99% reduction of tumor size.

In any of the embodiments described herein, the method results in at least about 70% reduction of tumor volume. In some embodiments, the method results in from about 70% to about 99% reduction of tumor volume. In some embodiments, the method results in at least about 80% reduction of tumor volume. In some embodiments, the method results in from about 80% to about 99% reduction of tumor volume. In some embodiments, the method results in about 70%, about 75%, about 80%, about 85%, about 90%, about 95%, or about 99% reduction of tumor volume.

In any of the embodiments described herein, the method results in at least about 40% reduction of cancer cell proliferation, tumor cell survival, tumor size, and/or tumor volume. In some embodiments, the method results in from about 40% to about 99% reduction of cancer cell proliferation, tumor cell survival, tumor size, and/or tumor volume. In any of the embodiments described herein, the method results in about 20%, about 25%, about 30%, about 35%, about 40%, about 45%, about 50%, about 55%, about 60%, about 65%, about 70%, about 75%, about 80%, about 85%, about 90%, about 95%, or about 99% reduction of cancer cell proliferation, tumor cell survival, tumor size, and/or tumor volume.

In any of the embodiments described herein, the method results in at least about 40% reduction of tumor size and/or tumor volume. In some embodiments, the method results in from about 40% to about 99% reduction of tumor size and/or tumor volume. In some embodiments, the method results in about 20%, about 25%, about 30%, about 35%, about 40%, about 45%, about 50%, about 55%, about 60%, about 65%, about 70%, about 75%, about 80%, about 85%, about 90%, about 95%, or about 99% reduction of tumor size and/or tumor volume.

In any of the methods described herein, the method provides an AUC (from day 0 to day 28) of at least about 90,000 ng*h/mL. In some embodiments, the method provides an AUC (from day 0 to day 28) of about 90,000 ng*h/mL to about 180,000 ng*h/mL, including about 90,000 ng*h/mL, about 100,000 ng*h/mL, about 110,000 ng*h/mL, about 120,000 ng*h/mL, about 130,000 ng*h/mL, about 140,000 ng*h/mL, about 150,000 ng*h/mL, about 160,000 ng*h/mL, about 170,000 ng*h/mL, and about 180,000 ng*h/mL.

In any of the methods described herein, the method provides a Cₘₐₓ of at least about 175 ng/mL. In some embodiments, the method provides a Cₘₐₓ of about 75 ng/mL to about 1500 ng/mL, including about 75 ng/mL, about 80 ng/mL, about 85 ng/mL, about 90 ng/mL, about 95 ng/mL, about 100 ng/mL, about 200 ng/mL, about 300 ng/mL, about 400 ng/mL, about 500 ng/mL, about 600 ng/mL, about 700 ng/mL, about 800 ng/mL, about 900 ng/mL, about 1000 ng/mL, about 1100 ng/mL, about 1200 ng/mL, about 1300 ng/mL, about 1400 ng/mL, and about 1500 ng/mL. In some embodiments, the method provides a Cₘₐₓ of about 1100 ng/mL.

In some embodiments, the temozolomide is administered at least once a day during the treatment cycle (e.g., a 28 day cycle). In some embodiments, the temozolomide is administered from one to five times a day during the treatment cycle. In some embodiments, the temozolomide is administered from one, two, three, four, or five times during the treatment cycle. In some embodiments, the temozolomide is administered once a day during the treatment cycle.

In some embodiments, the temozolomide is administered on at least about two consecutive days during the treatment cycle (e.g., a 28 day cycle). In some embodiments, the temozolomide is administered on from about two to about seven consecutive days during the treatment cycle. In some embodiments, the temozolomide is administered on about two, about three, about four, about five, about six, or about seven consecutive days during the treatment cycle. In some embodiments, the temozolomide is administered on about five consecutive days during the treatment cycle.

In some embodiments, the temozolomide is administered on Day 1, Day 2, Day 3, Day 4, and Day 5 of the treatment cycle (e.g., a 28 day cycle). In some embodiments, the temozolomide is administered on Day 2, Day 3, Day 4, Day 5, and Day 6 of the treatment cycle. In some embodiments, the temozolomide is administered on Day 3, Day 4, Day 5, Day 6, and Day 7 of the treatment cycle. In some embodiments, the temozolomide is administered on Day 4, Day 5, Day 6, Day 7, and Day 8 of the treatment cycle. In some embodiments, the temozolomide is not administered on Days 6-28, Days 6-29, Days 6-30, or Days 6-31 of the treatment cycle.

In any of the embodiments described herein, the single dose of the temozolomide is about 50 mg/m². In some embodiments, the single dose of the temozolomide is about 75 mg/m². In some embodiments, the single dose of the temozolomide is about 150 mg/m². In some embodiments, the single dose of the temozolomide is at least about 175 mg/m². In some embodiments, the single dose of the temozolomide is about 200 mg/m².

The Response Assessment in Neuro-Oncology criteria (RANO) is used to assess response to first-line treatment of glioblastoma. See, Leao, D. J. et al., American Journal of Neuroradiology 2019, 1-11. The RANO criteria divides response into four types of response based on imaging (MRI) and clinical features: (1) complete response (CR); (2) partial response (PR); (3) stable disease (SD); and (4) progression. For this assessment, a measurable disease is defined as bidimensional contrast-enhancing lesions with clearly defined margins, with 2 perpendicular diameters of at least 10 mm, visible on ≥2 axial slices. The nonmeasurable disease is defined as either unidimensional measurable lesions, masses with margins not clearly defined as frequently noted in the surgical margins, or lesions with maximal perpendicular diameters of <10 mm.

The criteria for complete response (CR) requires all of the following: complete disappearance of all enhancing measurable and nonmeasurable disease sustained for at least 4 weeks; no new lesions; and stable or improved nonenhancing (T2/FLAIR) lesions.

The criteria for partial response (PR) requires all of the following: ≥50% decrease, compared with baseline; the sum of products of perpendicular diameters of all measurable enhancing lesions sustained for at least 4 weeks; no progression of nonmeasurable disease; and no new lesions; stable or improved nonenhancing (T2/FLAIR) lesions imaging features.

The stable disease (SD) occurs if the patient does not qualify for complete response, partial response, or progression and requires the following: stable nonenhancing (T2/FLAIR) lesions.

Progression is defined by any of the following: ≥25% increase in the sum of the products of perpendicular diameters of enhancing lesions (compared with baseline if no decrease); a significant increase in T2/FLAIR nonenhancing lesions; the appearance of any new lesions; clear progression of nonmeasurable lesions; or definite clinical deterioration not attributable to other causes apart from the tumor.

In any of the embodiments described herein, the method achieves a Response Assessment in Neuro-Oncology Criteria (RANO) definition of complete response (CR) in the subject. In any of the embodiments described herein, the method achieves a Response Assessment in Neuro-Oncology Criteria (RANO) definition of partial response (PR) in the subject. In any of the embodiments described herein, the method achieves a Response Assessment in Neuro-Oncology Criteria (RANO) definition of stable disease (SD) in the subject.

In any of the embodiments described herein, complete response (CR), partial response (PR), and stable disease (SD) in the subject is after treatment with the bromodomain inhibitor. In any of the embodiments described herein, complete response (CR), partial response (PR), and stable disease (SD) in the subject is after treatment with the bromodomain inhibitor and a chemotherapy drug, such as temozolomide. In any of the embodiments described herein, complete response (CR), partial response (PR), and stable disease (SD) in the subject is after treatment with the bromodomain inhibitor, a chemotherapy drug, such as temozolomide, and radiation therapy.

### IV. Definitions

The following definitions are provided to facilitate understanding of certain terms used throughout this specification.

Technical and scientific terms used herein have the meanings commonly understood by one of ordinary skill in the art, unless otherwise defined. Any suitable materials and/or methodologies known to those of ordinary skill in the art can be utilized in carrying out the methods described herein.

As used in the description of the invention and the appended claims, the singular forms “a”, “an” and “the” are used interchangeably and intended to include the plural forms as well and fall within each meaning, unless the context clearly indicates otherwise. Also, as used herein, “and/or” refers to and encompasses any and all possible combinations of one or more of the listed items, as well as the lack of combinations when interpreted in the alternative (“or”).

As used herein, “about” will be understood by persons of ordinary skill in the art and will vary to some extent on the context in which it is used. If there are uses of the term which are not clear to persons of ordinary skill in the art given the context in which it is used, “about” will mean up to plus or minus 10% of the particular term.

The term “administering” as used herein includes prescribing for administration as well as actually administering, and includes physically administering by the subject being treated or by another.

As used herein “subject, “patient,” or “individual” refers to any subject, patient, or individual, and the terms are used interchangeably herein. In this regard, the terms “subject,” “patient,” and “individual” includes mammals, and, in particular humans. When used in conjunction with “in need thereof,” the term “subject,” “patient,” or “individual” intends any subject, patient, or individual having or at risk for a specified symptom or disorder.

As used herein, the phrase “therapeutically effective” or “effective” in context of a “dose” or “amount” means a dose or amount that provides the specific pharmacological effect for which the compound or compounds are being administered. It is emphasized that a therapeutically effective amount will not always be effective in achieving the intended effect in a given subject, even though such dose is deemed to be a therapeutically effective amount by those of skill in the art. For convenience only, exemplary dosages are provided herein. Those skilled in the art can adjust such amounts in accordance with the methods disclosed herein to treat a specific subject suffering from a specified symptom or disorder. The therapeutically effective amount may vary based on the route of administration and dosage form.

The terms “treatment,” “treating,” or any variation thereof includes reducing, ameliorating, or eliminating (i) one or more specified symptoms and/or (ii) one or more symptoms or effects of a specified disorder. The terms “prevention,” “preventing,” or any variation thereof includes reducing, ameliorating, or eliminating the risk of developing (i) one or more specified symptoms and/or (ii) one or more symptoms or effects of a specified disorder.

### EXAMPLES

As used in the following examples, Compound A or trotabresib refers to 4-[2-(cyclopropylmethoxy)-5-methylsulfonylphenyl]-2-methylisoquinolin-1-one.

### Example 1—Compound a in Patients with High-Grade Gliomas: A Phase 1 Open-Label ‘Window of Opportunity’ Study

The primary objectives of the study in this example were determination of tumor tissue concentration and plasma pharmacokinetics (PK) of Compound A. The secondary and exploratory objectives included safety, antitumor activity, cerebrospinal fluid concentration, and pharmacodynamics (PD).

The study described in this example (CC-90010-GBM-001 study/NCT04047303) enrolled patients with progressive or recurrent astrocytoma or recurrent glioblastoma who have failed radiation and chemotherapy, and who are candidates for surgical tumor resection as part of their salvage regimen.

Patients were treated with Compound A at 30 mg daily on Day 1 to Day 4 prior to undergoing indicated tumor resection and to maintain platelet count above the minimum of 100,000/mL recommended for surgery (Part A, preoperative treatment). Following recovery from surgery and a minimum of 4 weeks from the first dose of Compound A, subjects who were fit to continue were treated with Compound A at 45 mg on a 4 days on/24 days off schedule (180 mg per 28-day cycle). Maintenance treatment continued until disease progression, unacceptable toxicity, or withdrawal of consent.

Twenty patients were enrolled. Baseline patient demographics and characteristics are shown in Table 1. Median age was 47 years, 14 patients (70%) were male and 6 (30%) female, and 12 patients (60%) had an ECOG PS of 0. Nineteen patients (95%) had glioblastoma and 1 patient had progressive diffuse astrocytoma. The enrolled population was heavily pretreated; all patients had previously received radiotherapy and 16 (80%) had received 2 or more previous systemic therapies. MGMT promoter was methylated in 7 patients (35%), unmethylated in 7 patients (35%), and unknown in 6 patients (30%). IDH mutation status was wild-type in 14 patients (70%), mutant in 5 patients (25%), and not otherwise specified in 1 patient (5%).

TABLE 1
Patient characteristics

| Patient characteristics | N = 20 | |
|---|---|---|
| Median age, years (range) | 47 | (33-75) |
| Sex, n (%) | | |
| Male | 14 | (70) |
| Female | 6 | (30) |
| ECOGPS, n (%) | | |
| 0 | 12 | (60) |
| 1 | 8 | (40) |
| Tumor type, n (%) | | |
| Glioblastoma | 19 | (95) |
| Diffuse astrocytoma | 1 | (5) |
| MGMT promoter methylation status, n (%)^{a} | | |
| Methylated | 7 | (35) |
| Unmethylated | 7 | (35) |
| Not reported | 6 | (30) |
| IDH mutation status, n (%)^{a} | | |
| Wild-type | 14 | (70) |
| Mutant | 5 | (25) |
| Not otherwise specified | 1 | (5) |
| Number of prior radiation therapies, n (%) | | |
| 1 | 19 | (95) |
| 2 | 1 | (5) |
| Number of prior systemic therapies, n (%) | | |
| 1 | 4 | (20) |
| 2 | 15 | (75) |
| >2 | 1 | (5) |
| Median time since initial diagnosis, years | 1.2 | (0.6-10.8) |
| (range) | | |

^{a}MGMT promoter methylation status and IDH mutation status were determined using archival surgically resected tumor tissue and assessed per standard methodology at each institution.
Abbreviations:
ECOGPS, Eastern Cooperative Oncology Group performance status
IDH, isocitrate dehydrogenase.

The blood PK, blood PD, tumor PK and tumor PD data were available for 20, 12, 20, and 11 patients, respectively. On cycle 1, day 1 (C1D1), geometric mean (GM) Cₘₐₓ was 392 ng/mL and GM AUC from 0 to 24 hours (AUC₀₋₂₄) was 5083 ng h/mL. On cycle 1, day 4 (C1D4), GM Cₘₐₓ was 720 ng/mL and GM AUC₀₋₂₄ was 11,250 ng h/mL. Median tₘₐₓ was 1.5 hours on C1D1 and 1.9 hours on C1D4, and mean t_{1/2} was 46 hours on C1D4.

The geometric mean peak of Compound A plasma concentration on Day 4 was 1.90 μM, and the median time to peak concentration was 1.75 hours. The median time from the day 4 dose of Compound A to resection was 23 hours (range, 4.6-31.3). Compound A was found to penetrate the BBB and was detected in brain tumor tissue. At the time of resection, the geometric mean concentrations of Compound A in plasma and brain tumor tissue were 1.02 and 0.74 μM, respectively. Geometric mean free Compound A concentration in brain tissue was calculated to be 0.028 μM. Geometric mean Compound A concentration in CSF (cerebrospinal fluid) was 0.14 μM, and the mean CSF:plasma ratio was 0.17. None of the brain tumor tissue samples for PK analyses was confirmed to comprise homogeneous contrast-enhancing or non-enhancing tumor tissue, precluding measurement in these subgroups.

Blood CCR1 (C-C chemokine receptor type 1) mRNA was reduced ≥50% after Dose 4 (45.8%, standard deviation f 28.5, of baseline). Blood HEXIM1 (hexamethylene bis-acetamide-inducible protein 1) mRNA was increased at 72-96 hours following first dose, and the percentage increase correlated with plasma concentration of Compound A at surgery. Tumor HEXIM1 RNA increased in 10 of 11 patients. Additionally, blood HEXIM1 mRNA expression was increased in all subjects at 72-96 hours following the first dose. Percentage HEXIM1 increase was related to plasma Compound A concentration at biopsy. Comparison of HEXIM1 expression in resected brain tumor tissue with expression in archival tissue showed that HEXIM1 mRNA was increased in 15 of 18 patients (group P=0.00093).

Eighteen patients (90%) had ≥1 treatment-related adverse event (TRAE). Nine patients (45%) had grade 3/4 TRAEs, most frequently thrombocytopenia (5 patients [25%]). Only one patient had serious TRAEs (hemiparesis and lethargy). Two patients died of intracranial hemorrhage unrelated to study drug.

Of 17 patients evaluable for anti-tumor response, 7 (41%) had stable disease per RANO criteria and 10 (59%) had PD per RANO criteria, with 3 ongoing beyond data cutoff at cycles 4-11. Median progression-free survival (PFS) was 1.9 months (95% CI, 1.4-3.3). Six month progression-free survival was 12%. This result reflects the poor prognosis for the population enrolled in this study, with the evaluable population comprising patients with heavily pretreated recurrent glioblastoma. Subgroup analysis found that patients with IDH wild-type disease had a median PFS of 3.0 months (95% CI 1.4-3.6) and a 6-month PFS rate of 16.3% (95% CI 2.7-40.4), while patients with IDH-mutant disease had a median PFS of 1.2 months (95% CI 0.4-1.9) and a 6-month PFS rate of 0% (95% CI 0-0). Nonetheless, the observed PFS in the IDH wild-type population is notable in that this subgroup typically has poorer prognosis than patients with IDH-mutant tumors. Outcomes in subgroups based on MGMT promoter methylation status were not evaluated due to the small number of patients with confirmed promoter methylation status (n=14). Two patients remain on treatment with stable disease at cycles 18 and 22; both of whom had IDH wild-type disease and a methylated MGMT promotor. Overall, Compound A showed good tumor tissue penetration, with PD signals of response, and was well tolerated.

At Cycle 1 Day 4, the concentration of Compound A was measured in resected brain tumor from 20 subjects at 6 to 24-hour post dose. At the time of resection (N=19), the mean (f SDev) concentration of Compound A in plasma was 397 f 77.78 ng/mL (geometric mean: 389.23 ng/mL; coefficient of variation: 20.1%). The mean ratio of compound concentration in the resected brain tissue to compound concentration in plasma is 0.84 f 0.25 for 19 of the 20 subjects. The median time from the last dose of Compound A to surgery was 23 hours (range, 4.6-31.3), and it may be reasonable to suggest that tissue concentrations of Compound A would be higher at plasma tmax (1.9 hours post-dose on day 4) if the brain serves as a rapidly equilibrating compartment. These results demonstrate that Compound A has high brain penetrance.

BBB penetration data for anticancer therapies in patients with glioblastoma are limited, with most studies performed in small populations. Of note, brain tumor tissue concentrations of Compound A are lower than in plasma, with a tissue:plasma ratio of 0.14. Brain tumor tissue penetration data for various small molecule targeted therapies shows marked variation in relative tissue concentrations across agents, with tissue:plasma ratios of median 0.71 for imatinib, mean 0.25 for olaparib, and 0.06-0.08 for erlotinib. In contrast, both lapatinib and gefitinib show accumulation in glioblastoma tumor tissue, with a mean tissue:plasma ratio of 13.0 for lapatinib and median gefitinib concentrations in tumor tissue and plasma of 4100 ng/g and 181 ng/mL, respectively (P<0.0001). However, neither lapatinib nor gefitinib have shown evidence of antitumor activity in glioblastoma despite the high drug concentrations reported in tumor tissue.

The results are detailed in FIGS. 1-6. In particular, FIG. 1 shows the CCR1% at 74 hours and IDH mutation relationship versus Days on Study for Example 1. This figure shows that subjects remaining on treatment longer were among those with the biggest % decrease in CCR1 at 74 Hrs. This figure also shows that subjects with shorter time on study <100 days exhibited less CCR1% decrease <50% at 74 hours.

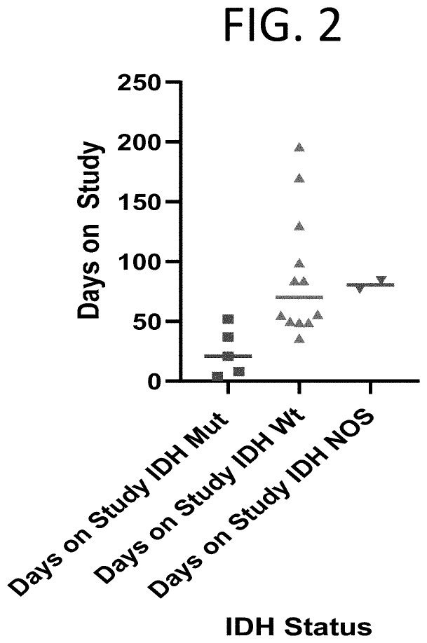

FIG. 2 shows the time on study versus IDH status for the subjects treated in Example 1. FIGS. 1 and 2 show that subjects have the IDH mutation were among those with shorter on-study time.

FIG. 3 shows the CCR1% at 74 hours and MGMT methylation status versus time on treatment for Example 1. This figure shows that subjects with methylated MGMT were among those remaining on treatment longer (3 of 4 over 100 days).

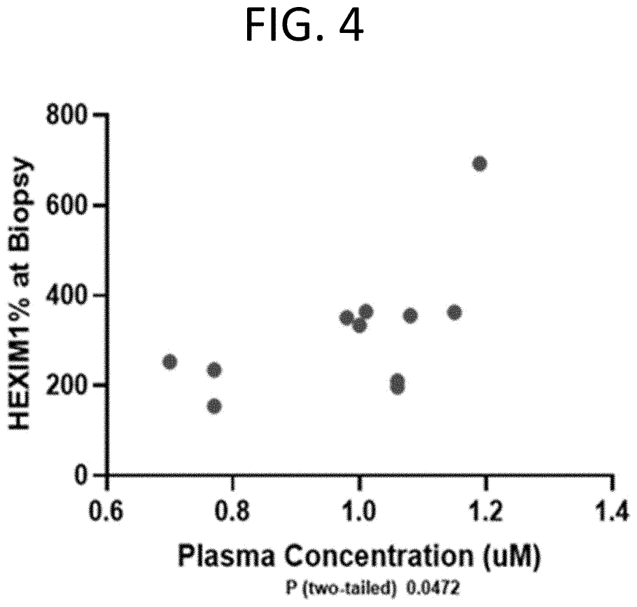

FIG. 4 shows the HEXIM1% at the time of biopsy versus plasma concentration in Example 1. This figure shows that the blood HEXIM1 mRNA was increased at 72-96 hours following first dose, and the percentage increase correlated with plasma concentration of Compound A at surgery.

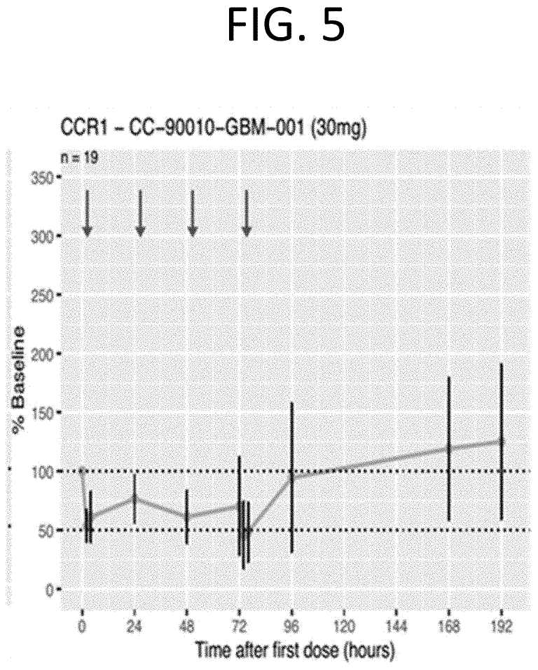

FIG. 5 shows the CCR1% versus time after the first dose in Example 1 at 30 mg of Compound A and is similar to the profile observed in Example 2 at the same dose and schedule. This figure shows that CCR1≥50% downregulation following first and last dose of ≥30 mg of Compound A with deepest suppression following last dose in cycle 1 in CC-90010-GBM-001.

Finally, FIG. 6 shows the CCR1% versus time after the first dose at 30 mg of Compound A and is similar to the profile observed in FIG. 5 at the same dose and schedule. This figure shows that CCR1≥50% downregulation following first and last dose of ≥30 mg of Compound A with deepest suppression following last dose in cycle 1 in CC-90010-GBM-002.

The results provided an understanding of (a) the pharmacokinetics and brain tumor tissue penetration of Compound A in patients with progressive or recurrent astrocytoma or recurrent glioblastoma; and (b) the safety, preliminary antitumor activity, and blood and tumor pharmacodynamics of Compound A in patients with progressive or recurrent astrocytoma or recurrent glioblastoma.

### Example 2—Compound a and Temozolomide in Patients with Newly Diagnosed Glioblastoma: Interim Results from a Phase 1B Dose-Finding Study

The primary objectives of the study in this example were to establish the safety, maximum tolerated dose (MTD), and recommended phase 2 dose (RP2D) of Compound A. The preliminary efficacy, pharmacokinetics, and pharmacodynamics were also investigated.

The study described in this example (CC-90010-GBM-002/NCT04324840) is a phase 1B dose-finding study investigating standard of care of Compound A in combination with TMZ with or without radiotherapy (RT) in subjects with newly diagnosed GBM. Part A of the study is escalating the oral doses of Compound A in combination with adjuvant TMZ to estimate the MTD and/or the RP2D of Compound A as adjuvant therapy and escalating the oral doses of Compound A in combination with concomitant TMZ+radiotherapy (RT) to estimate the MTD and/or the RP2D of Compound A as concomitant therapy. Part B of the study is the safety and efficacy of Compound A administered at or below the MTD determined for the adjuvant therapy (AT) and concomitant therapy (CT).

For adjuvant therapy, Compound A and TMZ was administered for 6 cycles, then Compound A as a monotherapy was administered in 28-day cycles until disease progression, start of a new anticancer therapy, or withdrawal of consent or physician's decision. For adjuvant therapy, Compound A was administered at 15, 30, and 45 mg 4 days on/24 days off and TMZ was administered at 150 or 200 mg/m² QD Days 1-5, followed by Compound A at 45 mg 4 days on/24 days off. For adjuvant therapy, part B, Compound A was administered at RP2D and TMZ was administered at 150 or 200 mg/m², followed by Compound A at 45 mg 4 days on/24 days off.

For concomitant therapy, Compound A, TMZ, and RT were administered for 6 weeks, then 4-week break, followed by adjuvant Compound A and TMZ for 6 cycles then Compound A monotherapy in 28-day cycles until disease progression, start of a new anticancer therapy, withdrawal of consent or physician's decision. For concomitant therapy, part A, Compound A was administered at 15 mg or 30 mg QD Days 1-4 (weeks 1,5), TMZ was administered at 75 mg/m²/day QD, and RT was administered at 2 Gy QD×5 days/week, for 42 days. This was then followed by a break in treatment therapy, e.g., from 1 to up to 4 weeks. Thereafter, adjuvant therapy was initiated by administering Compound A at 30 mg or 45 mg and TMZ at 150 or 200 mg/m² QD Days 1-5 of a 28-day cycle. Adjuvant therapy cycles were repeated from 2 to 6 times. After adjuvant therapy, was a monotherapy treatment. Monotherapy comprised administering Compound A at 45 mg 4 days on/24 days off.

Thus, the treatment groups were concomitant therapy (Compound A+radiotherapy+temozolomide followed by adjuvant therapy and then Compound A monotherapy) and adjuvant therapy (Compound A+temozolomide followed Compound A monotherapy).

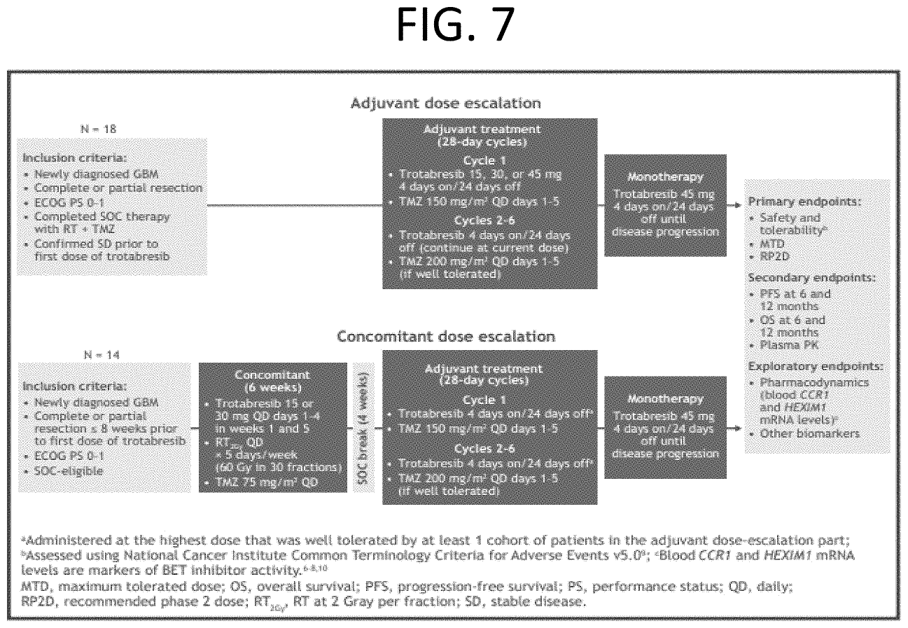

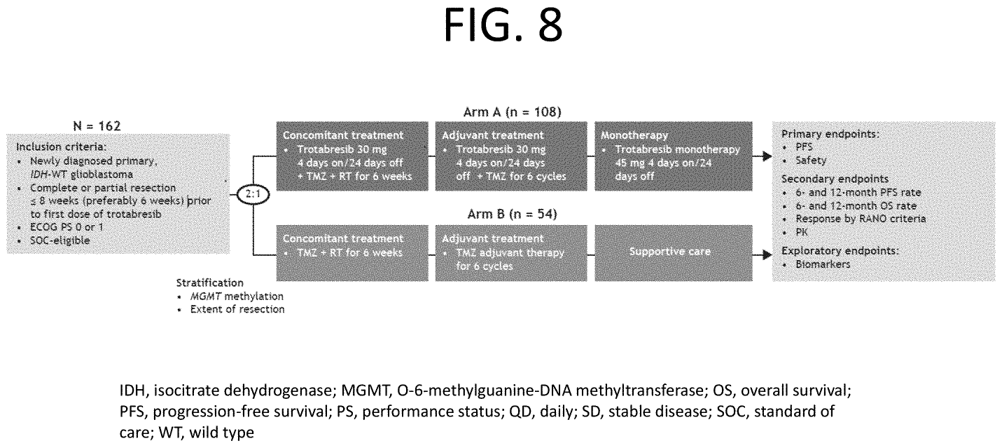

FIG. 7 shows the adjuvant dosing and concomitant dosing schedules for Compound A in Part A of the study. FIG. 8 shows the adjuvant dosing and concomitant dosing schedules for Compound A in Part B of the study.

The interim results for adjuvant Compound A+temozolomide are described. For adjuvant therapy, patients received Compound A at 15, 30, or 45 mg daily (4 days on/24 days off)+temozolomide administered per label for 6 cycles (150 mg/m² at Cycle 1 then 200 mg/m²; QD for 5 days), followed by Compound A at 45 mg monotherapy daily (4 days on/24 days off).

For concomitant therapy dose escalation starts, patients received Compound A 15 mg, 30 mg, or 45 mg daily (4 days on/24 days off schedule) in combination with concomitant temozolomide (75 mg/m² QD for 7 days for 42 days) and RT (fractionated focal irradiation at 2 Gy per fraction QD 5 days per week for 42 days).

For Part A, eighteen patients were enrolled in the adjuvant cohort and received adjuvant Compound A 15 mg/day (n=5), 30 mg/day (n=6), or 45 mg/day (n=7) 4 days on/24 days off plus TMZ followed by Compound A monotherapy 45 mg/day 4 days on/24 days off. Fourteen patients were enrolled in the concomitant cohort and received concomitant Compound A 15 mg/day (n=6) or 30 mg/day (n=8) 4 days on/24 days off in combination with TMZ and RT, followed by adjuvant Compound A 30 mg/day 4 days on/24 days off and Compound A monotherapy 45 mg/day 4 days on/24 days off. The demographics and characteristics of treated patients are described in the below table.

TABLE 2
Patient characteristics for Part A

| | Adjuvant | Concomitant |
|---|---|---|
| | cohort | cohort |
| Patient characteristics | (n = 18) | (n = 14) |
| Median age (range), years | 53 (31-70) | 56 (29-71) |
| Sex, n (%) | | |
| Male | 12 (67) | 8 (57) |
| Female | 6 (33) | 6 (43) |
| ECOGPS, n (%) | | |
| 0 | 14 (78) | 5 (36) |
| 1 | 4 (22) | 9 (64) |
| Type of resection, n (%) | | |
| Complete | 10 (56) | 9 (64) |
| Partial | 8 (44) | 5 (36) |
| MGMT promoter methylation status, n (%) | | |
| Methylated | 6 (33) | 7 (50) |
| Unmethylated | 10 (56) | 3 (21) |
| Not reported | 0 | 4 (29) |
| IDH mutation status, n (%) | | |
| Wild-type | 17 (94) | 11 (79) |
| Mutant | 1 (6) | 2 (14) |
| Not reported | 0 | 1 (7) |

For Part B, as of Aug. 1, 2022, 29 patients have enrolled in arm A and 15 patients have enrolled in arm B. The demographics and characteristics of treated patients are described in the below table.

TABLE 3
Patient characteristics for Part B

| | Arm A | Arm B |
|---|---|---|
| Patient characteristics | (n = 29) | (n = 15) |
| Median age (range), years | 57 (35-70) | 62 (44-68) |
| Sex, n (%) | | |
| Male | 20 (69) | 8 (53) |
| Female | 9 (31) | 7 (47) |
| Karnofsky PS, n (%) | | |
| 100 | 12 (41) | 7 (47) |
| 90 | 13 (45) | 5 (33) |
| 80 | 4 (14) | 2 (13) |
| 70 | 0 | 1 (7) |
| Type of resection, n (%) | | |
| Complete | 18 (62) | 11 (73) |
| Partial | 11 (38) | 4 (27) |
| MGMT promoter methylation status, n (%) | | |
| Methylated | 14 (48) | 7 (47) |
| Unmethylated | 15 (52) | 8 (53) |
| Not reported | 0 | 0 |
| IDH mutation status, n (%) | | |
| Wild-type | 29 (100) | 15 (100) |
| Mutant | 0 | 0 |
| Not reported | 0 | 0 |

Exposure of Compound A increased proportionally with dose. Day 4 time to peak Compound A concentration was 0.5-2.0 hours; mean terminal half-life was 60-70 hours. Day 4 blood CCR1 RNA 2-4 hours post-dose was downregulated partially in the 15-mg group and ≥50% in the 30-mg group.

FIG. 6 shows the CCR1% versus time after the first dose in Example 2 at 30 mg of Compound A and is similar to the profile observed in Example 1 at the same dose and schedule. This figure shows that CCR1≥50% downregulation following first and last dose of ≥30 mg of Compound A with deepest suppression following last dose in cycle 1.

For Part A, as of the study data cutoff date, any-grade treatment-related adverse events (TRAEs) were reported in 17 (94%) and 14 (100%) patients in the overall adjuvant and concomitant cohorts, respectively. Grade 3/4 TRAEs were reported in 10 (56%) patients and 9 (64%) patients in the adjuvant and concomitant cohorts, respectively. There were no treatment-related deaths. In the adjuvant cohort: (a) thrombocytopenia was the most common TRAE, reported in 13 (72%) patients at any grade and in 9 (50%) patients at grade 3/4; and (b) any-grade gastrointestinal TRAEs were reported in 14 patients (78%); one patient had grade 3/4 diarrhea. In the concomitant cohort: (a) nausea was the most frequent any-grade TRAE, reported in 11 (79%) patients; (b) thrombocytopenia was reported in 10 (71%) patients at any grade and in 7 (50%) patients at grade 3/4; and (c) gastrointestinal TRAEs were reported in 13 (93%) of patients, with a grade 3/4 TRAE of diarrhea reported in 1 (7%) patient. No new safety signals were reported during extended follow-up. Treatment emergent AEs (TEAEs) leading to treatment discontinuation were reported in 2 patients (11%) in the adjuvant cohort (1 in the 45 mg/day group due to a TEAE unrelated to study drug, 1 in the 30 mg/day group due to dose limiting toxicity of thrombocytopenia) and 1 patient (7%) in the concomitant cohort (30 mg/day group due to TEAE unrelated to study drug).

For Part B, TRAEs related to Compound A were reported in 24 (86%) patients in arm A, most commonly thrombocytopenia (n=13; 46%), nausea (n=13; 46%), and diarrhea (n=12; 43%). Grade 3/4 TRAEs related to Compound A were reported in 8 (29%) of patients, most commonly neutropenia and thrombocytopenia (n=5, 18% each). TRAEs related to TMZ were reported in 21 (75%) and 11 (73%) patients in arms A and B, respectively; most commonly nausea and thrombocytopenia (n=13, 46% each) in arm A, and thrombocytopenia (n=6, 40%) in arm B. TRAE leading to treatment discontinuation was reported in 1 patient (4%, arm A).

For Part A, as of the Aug. 1, 2022, data cutoff, median duration of treatment was 37 weeks (range: 6-96) in the adjuvant cohort and 46 weeks (range: 10-74) in the concomitant cohort. Five (28%) and 4 (29) patients in the adjuvant and concomitant cohorts, respectively, remained on treatment. The progression-free survival (PFS) rate at 6 months was 57.8% (95% CI, 31.1-77.3) in the adjuvant cohort. PFS rate at 6 months was 69.2% (95% CI, 37.3-87.2) in the concomitant cohort. Median PFS was 7.7 months (95% CI, 3.9—not estimable [NE]) in the adjuvant cohort and 12.3 months (95% CI, 5.2-14.0) in the concomitant cohort. For the best overall response, the adjuvant cohort had the following responses: CR=6% (n=1), SD=78% (n=14), PD=11% (n=2), NE=6% (n=1). For the best overall response, the concomitant cohort had the following responses: CR=7% (n=1), SD=79% (n=11), PD=7% (n=1), NE=(7% (n=1).

The below table shows the plasma PK data in this study. Time to peak Compound A concentration on day 4 was 0.5-4.0 hours and mean terminal half-life was ˜60-70 hours. The PK parameters in Part B were consistent with Part A (dose escalation) and prior Compound A studies (e.g., Example 1).

TABLE 4
Pharmacokinetics of Compound A

| Parameter | | GBM-002 (Part A) | | GBM-002 (Part B) | |
|---|---|---|---|---|---|
| (Unit) | Visit | (n = 6) | | (n = 17) | |
| Cₘₐₓ | D1 | 306 | (56) | 350 | (44) |
| (ng/mL) | LD | 822 | (27) | 740 | (19) |
| tₘₐₓ (h) | D1 | 2.0 | (1.5-4.0)) | 1.5 | (0.5-4.0) |
| | LD | 1.5 | (1.5-2.0) | 1.50 | (1.0-4.0) |
| AUC₂₄ | D1 | 4580 | (26) | 4889 | (24) |
| (ng × h/mL) | LD | 13,947 | (24) | 12,741 | (18) |
| T_{1/2} (h) | LD | 68 | (33) | 58 | (58) |
| AUC_{inf} | LD | 49,786 | (63) | 48,678 | (58) |
| (ng × h/mL) | | | | | |

| | | | |
|---|---|---|---|
| AI Cₘₐₓ | NA | 2.69 | 2.11 |
| AI AUC₂₄ | | 3.05 | 2.61 |

Data presented as geometric mean (geometric CV %) for AUC and Cₘₐₓ, mean (SD) for t_{1/2}, and median (range) for tₘₐₓ. AI AUC, accumulation index ratio of exposure measured at Day 4 to that after Day 1; AI Cₘₐₓ, accumulation index ratio of maximum observed concentration at Day 4 to that after Day 1; AUC₂₄, area under the concentration-time curve within a 24-hour dosing interval; Cₘₐₓ, maximum observed concentration; NA, not applicable; t_{1/2}, apparent terminal phase half-life.

Adjuvant Compound A plus TMZ and concomitant Compound A plus TMZ and RT in patients with newly diagnosed GBM continued to be well tolerated in long-term follow-up with no new safety signals. The safety profile remains consistent with previous studies of Compound A monotherapy and adjuvant TMZ. As of the study data cutoff date, median total duration of treatment was 37 weeks and 46 weeks in the adjuvant and concomitant cohorts, respectively. Five (28%) and 4 (29%) patients in the adjuvant and concomitant cohorts, respectively, remained on treatment. Six-month PFS rates were 57.8% and 69.2% in the adjuvant and concomitant cohorts, respectively. Compound A plasma PK in part B were similar to those observed in clinical trials investigating Compound A monotherapy. Coadministration with RT and TMZ did not appear to impact Compound A PK.

### Example 3—Preparation of Spray-Dried Dispersions of Compound a with HPMCAS-H

Spray-dried dispersions (SDD) were prepared by mixing a solution of Compound A in (90:10) acetone:water with hydroxypropylmethylcellulose acetate succinate (HPMCAS-HG) in ratios of Compound A:polymer of either 1:1, 1:2.85, or 1:3, followed by spray-drying each preparation using a custom bench scale Lab Spray Dryer (Bend Research BLD-35; process parameters: inlet T 84-94° C.; outlet T 40-42° C.; atomization pressure 120 psi; nozzle—pressure swirl/Schlick 2.0; solution spray rate 25 g/min; airflow 475 g/min; setup: open loop).

### Example 4—Immediate Release Tablet Containing 10 mg of Compound A

An immediate release tablet containing 10 mg of Compound A was generally prepared as follows. The raw materials described in Table 5 (below) were blended, sieved, and blended again prior to granulation. The blended raw materials were granulated using a dry granulation process. Then the dry granulated materials were blended with the extragranular materials. The blended materials were compressed into tablets using 6 mm SRC (standard round concave) tooling.

TABLE 5

| | % of | Amount per |
|---|---|---|
| Ingredient | tablet | tablet (mg) |
| Intragranular | | |
| SDD 1:3 Compound A:HPMCAS-HG | 33.33 | 40.0 |
| Microcrystalline cellulose (Avicel PH 101) | 29.34 | 35.2 |
| Lactose (310) | 29.33 | 35.2 |
| Croscarmellose sodium (Ac-Di-Sol) | 6.00 | 7.2 |
| Colloidal Silicon Dioxide (Cabosil M5P) | 0.50 | 0.6 |
| Magnesium stearate | 0.50 | 0.6 |
| Extragranular | | |
| Colloidal Silicon Dioxide (Cabosil M5P) | 0.50 | 0.6 |
| Magnesium stearate | 0.50 | 0.6 |
| Total | 100.00 | 120.0 |

### Example 5—Immediate Release Tablet Containing 100 mg of Compound A

One version of an immediate release tablet containing 100 mg of Compound A was generally prepared as follows. The raw materials described in Table 6 (below) were blended, sieved, and blended again prior to granulation. The blended raw materials were granulated using a dry granulation process. Then the dry granulated materials were blended with the extragranular materials. The blended materials were compressed into tablets using concave modified oval tooling (9.1 mm×18.1 mm).

TABLE 6

| | % of | Amount per |
|---|---|---|
| Ingredient | tablet | tablet (mg) |
| Intragranular | | |
| SDD 1:3 Compound A:HPMCAS-HG | 50.00 | 400.0 |
| Microcrystalline cellulose (Avicel PH 101) | 21.00 | 168.0 |
| Lactose (310) | 21.00 | 168.0 |
| Croscarmellose sodium (Ac-Di-Sol) | 6.00 | 48.0 |
| Colloidal Silicon Dioxide (Cabosil M5P) | 0.50 | 4.0 |
| Magnesium stearate | 0.50 | 4.0 |
| Extragranular | | |
| Colloidal Silicon Dioxide (Cabosil M5P) | 0.50 | 4.0 |
| Magnesium stearate | 0.50 | 4.0 |
| Total | 100.00 | 800.0 |

### Example 6—Immediate Release Tablet Containing 200 mg of Compound A

One version of an immediate release tablet containing 200 mg of Compound A is prepared as follows. The raw materials described in Table 7 (below) are blended, sieved, and blended again prior to granulation. The blended raw materials are granulated using a dry granulation process. Then the dry granulated materials are blended with the extragranular materials. The blended materials are the compressed into tablets.

TABLE 7

| | % of | Amount per |
|---|---|---|
| Ingredient | tablet | tablet (mg) |
| Intragranular | | |
| SDD 1:1 Compound A:HPMCAS-HG | 50.00 | 400.0 |
| Microcrystalline cellulose (Avicel PH 101) | 21.00 | 168.0 |
| Lactose (310) | 21.00 | 168.0 |
| Croscarmellose sodium (Ac-Di-Sol) | 6.00 | 48.0 |
| Colloidal Silicon Dioxide (Cabosil M5P) | 0.50 | 4.0 |
| Magnesium stearate | 0.50 | 4.0 |
| Extragranular | | |
| Colloidal Silicon Dioxide (Cabosil M5P) | 0.50 | 4.0 |
| Magnesium stearate | 0.50 | 4.0 |
| Total | 100.00 | 800.0 |

While certain embodiments have been illustrated and described, it should be understood that changes and modifications can be made therein in accordance with ordinary skill in the art without departing from the technology in its broader aspects as defined in the following claims.

The embodiments, illustratively described herein may suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the claimed technology. Additionally, the phrase “consisting essentially of” will be understood to include those elements specifically recited and those additional elements that do not materially affect the basic and novel characteristics of the claimed technology. The phrase “consisting of” excludes any element not specified.

The present disclosure is not to be limited in terms of the particular embodiments described in this application. Many modifications and variations can be made without departing from its spirit and scope, as will be apparent to those skilled in the art.

All publications, patent applications, issued patents, and other documents referred to in this specification are herein incorporated by reference as if each individual publication, patent application, issued patent, or other document was specifically and individually indicated to be incorporated by reference in its entirety. Definitions that are contained in text incorporated by reference are excluded to the extent that they contradict definitions in this disclosure.

Other embodiments are set forth in the following claims

What is claimed is:

1. A method of treating glioblastoma multiforme (GBM) in a newly diagnosed GBM subject in need thereof comprising:
(i) administering to the newly diagnosed GBM subject, a concomitant treatment of radiotherapy with temozolomide and a compound having the structure of Formula (I), 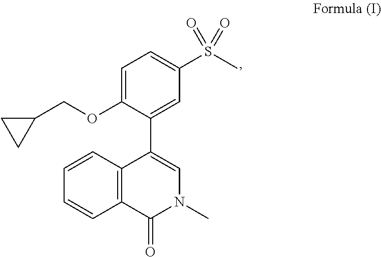 [SMILES: Cn1cc(-c2cc(S(C)(=O)=O)ccc2OCC2CC2)c2ccccc2c1=O]
or a pharmaceutically acceptable salt thereof; thereafter
(ii) adjunctively administering temozolomide and the compound of Formula (I) or a pharmaceutically acceptable salt thereof without radiotherapy; and thereafter
(iii) administering the compound of Formula (I) or a pharmaceutically acceptable salt thereof as a monotherapy.

2. The method of claim 1, wherein step (i) comprises a 42-day treatment regimen comprising administering the compound of Formula (I) or a pharmaceutically acceptable salt thereof on Days 1-4 and Days 35-39.

3. The method of claim 2, wherein step (i) comprises a 42-day treatment regimen comprising administering 15 mg or 30 mg of the compound of Formula (I) or a pharmaceutically acceptable salt on Days 1-4 and Days 35-39.

4. The method of claim 1, wherein step (i) comprises a 42-day treatment regimen comprising administering temozolomide once daily for the 42 days.

5. The method of claim 4, wherein step (i) comprises a 42-day treatment regimen comprising administering 75 mg/m² of temozolomide once daily for the 42 days.

6. The method of claim 1, wherein step (ii) comprises a 28-day cycle comprising administering the compound of Formula (I) or a pharmaceutically acceptable salt thereof on Days 1-4.

7. The method of claim 6, wherein step (ii) comprises a 28-day cycle comprising administering 15 mg, 30 mg, or 45 mg of compound of Formula (I) or a pharmaceutically acceptable salt thereof on Days 1-4.

8. The method of claim 1, wherein step (ii) comprises a 28-day cycle comprising administering temozolomide on Days 1-5.

9. The method of claim 8, wherein step (ii) comprises a 28-day cycle comprising administering 150 mg/m² of temozolomide on Days 1-4.

10. The method of claim 1 wherein step (ii) comprises a 28 day cycle repeating from 2 to 6 times, and wherein 15 mg, 30 mg, or 45 mg of the compound of Formula (I) or a pharmaceutically acceptable salt is administered on Days 1-4 of cycles 2-6 and wherein temozolomide is administered at a dose of 200 mg/m² on Days 1-5 of cycles 2-6.

11. The method of claim 1, wherein step (iii) comprises a 28-day cycle comprising administering the compound of Formula (I) or a pharmaceutically acceptable salt thereof on Days 1-4.

12. The method of claim 11, wherein step (iii) comprises a 28-day cycle comprising administering 45 mg of compound of Formula (I) or a pharmaceutically acceptable salt thereof on Days 1-4.

13. The method of claim 11, wherein step (iii) comprises a 28-day cycle comprising administering the compound of Formula (I) or a pharmaceutically acceptable salt thereof is over one, two, three, four, five, six, seven, eight, nine, or ten cycles.

14. A method of treating glioblastoma multiforme (GBM) in a subject in need thereof comprising adjunctively administering temozolomide and the compound of having the structure of Formula (I), 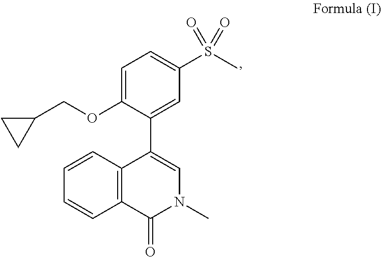 [SMILES: Cn1cc(-c2cc(S(C)(=O)=O)ccc2OCC2CC2)c2ccccc2c1=O]
or a pharmaceutically acceptable salt thereof without radiotherapy, and
wherein administering the temozolomide and the compound of Formula (I) are in a 28 day cycle.

15. The method of claim 14, wherein administering the compound of Formula (I) or a pharmaceutically acceptable salt thereof is on Days 1-4.

16. The method of claim 15, wherein 15 mg, 30 mg, or 45 mg of the compound of Formula (I) or a pharmaceutically acceptable salt is administered on Days 1-4.

17. The method of claim 14, wherein administering temozolomide is on Days 1-5.

18. The method of claim 17, wherein 150 mg/m² of temozolomide is administered on Days 1-4 of a 28-day cycle.

19. The method of claim 14, wherein the 28-day cycle repeats 2 to 6 times, and wherein 15 mg, 30 mg, or 45 mg of the compound of Formula (I) or a pharmaceutically acceptable salt is administered on Days 1-4 of cycles 2-6, and wherein temozolomide is administered at a dose of 200 mg/m² on Days 1-5 of cycles 2-6.

20. A method of treating glioblastoma multiforme (GBM) in a subject in need thereof comprising administering a monotherapy treatment of 45 mg of a compound having the structure of Formula (I), 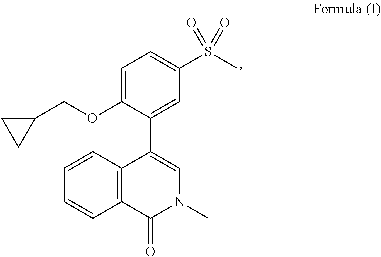 [SMILES: Cn1cc(-c2cc(S(C)(=O)=O)ccc2OCC2CC2)c2ccccc2c1=O]
or a pharmaceutically acceptable salt thereof on Days 1-4 of a 28 day cycle, and
wherein the compound is administered in an amount sufficient to result in a mean ratio of compound concentration in the resected brain tissue to compound concentration in plasma of from about 0.50 to about 1.50.

21. The method of claim 20, wherein administering the compound of Formula (I) or a pharmaceutically acceptable salt thereof is over more than one 28 day cycle.

22. The method of claim 22, wherein administering the compound of Formula (I) or a pharmaceutically acceptable salt thereof is over two, three, four, five, six, seven, eight, nine, or ten 28 day cycles.

23. The method of claim 1, wherein the compound of Formula (I) or a pharmaceutically acceptable salt thereof is administered in an amount sufficient to result in a mean ratio of compound concentration in the resected brain tissue to compound concentration in plasma of from about 0.50 to about 1.50.

24. The method of claim 14, wherein the compound of Formula (I) or a pharmaceutically acceptable salt thereof is administered in an amount sufficient to result in a mean ratio of compound concentration in the resected brain tissue to compound concentration in plasma of from about 0.50 to about 1.50.

25. The method of claim 1, wherein the glioblastoma is O-6-methylguanine-DNA methyltransferase (MGMT)-positive glioblastoma.

26. The method of claim 14, wherein the glioblastoma is O-6-methylguanine-DNA methyltransferase (MGMT)-positive glioblastoma.

27. The method of claim 20, wherein the glioblastoma is O-6-methylguanine-DNA methyltransferase (MGMT)-positive glioblastoma.

28. The method of claim 1, wherein the glioblastoma is O-6-methylguanine-DNA methyltransferase (MGMT)-negative glioblastoma.

29. The method of claim 14, wherein the glioblastoma is O-6-methylguanine-DNA methyltransferase (MGMT)-negative glioblastoma.

30. The method of claim 20, wherein the glioblastoma is O-6-methylguanine-DNA methyltransferase (MGMT)-negative glioblastoma.

31. The method of claim 25, wherein the MGMT-positive glioblastoma is determined by methylation status of the gene, mRNA expression, and/or protein expression.

32. The method of claim 28, wherein the MGMT negative glioblastoma is determined by methylation status of the gene, mRNA expression, and/or protein expression.

33. The method of claim 1, wherein the glioblastoma has no or a low level O-6-methylguanine-DNA methyltransferase (MGMT) expression.

34. The method of claim 1, wherein the compound of Formula (I), or a pharmaceutically acceptable salt thereof, is adapted for oral administration.

35. The method of claim 34, wherein the compound of Formula (I), or a pharmaceutically acceptable salt thereof, is in the form of a tablet, pill, sachet, or capsule of hard of soft gelatin.

36. The method of claim 1, wherein the compound of Formula (I), or a pharmaceutically acceptable salt thereof, has a terminal half-life of at least about 60 hours.

37. The method of claim 1, wherein the method provides a platelet count of at least about 100 [*10⁹/L] in the subject.

38. The method of claim 1, wherein the method provides for a 50% decline from C-C motif chemokine receptor 1 (CCR1) baseline in the subject.

39. The method of claim 38, wherein the method provides for a 50% decline from C-C motif chemokine receptor 1 (CCR1) baseline in the subject when measured 74 hours post first dose of the compound.

40. The method of claim 1, wherein the method provides:
(a) an AUC (from day 0 to day 28) of at least about 90,000 ng*h/mL;
(b) an AUC (from day 0 to day 28) of from about 90,000 ng*h/mL to about 180,000 ng*h/mL
(c) a Cₘₐₓ of at least about 175 ng/mL;
(d) a Cₘₐₓ of from about 75 ng/mL to about 1500 ng/mL; and/or
(e) a Cₘₐₓ of about 1100 ng/mL.

41. The method of claim 1, wherein:
(a) the method results in at least about 70% reduction of cancer cell proliferation, tumor cell survival, tumor size, and/or tumor volume;
(b) the method results in from about 70% to about 99% reduction of cancer cell proliferation, tumor cell survival, tumor size, and/or tumor volume;
(c) the method results in at least about 80% reduction of cancer cell proliferation, tumor cell survival, tumor size, and/or tumor volume;
(d) the method results in from about 80% to about 99% reduction of cancer cell proliferation, tumor cell survival, tumor size, and/or tumor volume; and/or
(e) the method results in about 70%, about 75%, about 80%, about 85%, about 90%, about 95%, or about 99% reduction of cancer cell proliferation, tumor cell survival, tumor size, and/or tumor volume.

42. The method claim 1, wherein:
(a) the method results in at least about 40% reduction of cancer cell proliferation, tumor cell survival, tumor size, and/or tumor volume;
(b) the method results in from about 40% to about 99% reduction of cancer cell proliferation, tumor cell survival, tumor size, and/or tumor volume;
and/or
(e) the method results in about 20%, about 25%, about 30%, about 35%, about 40%, about 45%, about 50%, about 55%, about 60%, about 65%, about 70%, about 75%, about 80%, about 85%, about 90%, about 95%, or about 99% reduction of cancer cell proliferation, tumor cell survival, tumor size, and/or tumor volume.

43. The method of claim 1, wherein:
(a) the method results in at least about 40% reduction of tumor size and/or tumor volume;
(b) the method results in from about 40% to about 99% reduction of tumor size and/or tumor volume; and/or
(c) the method results in about 20%, about 25%, about 30%, about 35%, about 40%, about 45%, about 50%, about 55%, about 60%, about 65%, about 70%, about 75%, about 80%, about 85%, about 90%, about 95%, or about 99% reduction of tumor size and/or tumor volume.

44. The method of claim 41, wherein the subject has a reduction of cancer cell proliferation, tumor cell survival, tumor size, and/or tumor volume after administration of the compound, or a pharmaceutically acceptable salt thereof after at least one dose of the compound of Formula (I) or a pharmaceutically acceptable salt thereof and/or after a 28 day cycle.

45. The method of claim 1, wherein the method achieves one or more of the following:
(a) a Response Assessment in Neuro-Oncology Criteria (RANO) definition of complete response (CR) in the subject;
(b) a Response Assessment in Neuro-Oncology Criteria (RANO) definition of partial response (PR) in the subject; and
(c) a Response Assessment in Neuro-Oncology Criteria (RANO) definition of stable disease (SD) in the subject.
